(19) United States
(12) Patent Application Publication
(10) Pub. No.: US 2014/0073604 A1
(43) Pub. Date: Mar. 13, 2014

(54) Ladder-Frame Polyether Conjugates

(71) Applicant: UNIVERSITY OF NORTH CAROLINA AT WILMINGTON, (US)

(72) Inventors: Andrea Bourdelais, Wilmington, NC (US); Daniel G. Baden, Wilmington, NC (US); Elizabeth Elliott, Wilmington, NC (US)

(73) Assignee: UNIVERSITY OF NORTH CAROLINA AT WILMINGTON, Wilmington, NC (US)

(21) Appl. No.: 13/840,296

(22) Filed: Mar. 15, 2013

Related U.S. Application Data
(62) Division of application No. 12/893,344, filed on Sep. 29, 2010.

(51) Int. Cl. A61K 47/22; G01N 33/50; A61K 31/69; A01N 25/00; A61K 31/4188; A01N 43/16; A01N 55/08; A01N 43/90; G01N 33/52; A61K 31/4375

(52) U.S. Cl. CPC A61K 47/22; G01N 33/52; G01N 33/502; A61K 31/69; A61K 31/4375; A61K 31/4188; A01N 43/16; A01N 55/08; A01N 43/90; A01N 25/00
USPC 514/64; 435/7.1; 435/7.9; 435/7.92; 435/29; 504/193; 504/245; 504/276; 504/292; 514/279; 514/283; 514/387; 514/450; 546/37; 546/48; 548/304.1; 548/405; 549/354

(57) ABSTRACT

Disclosed are compounds that are conjugates of ladder frame polyether compounds and biologically active compounds or research compounds, pharmaceutical formulations comprising the conjugates, and methods of transporting the conjugates across biological membranes.

### BACKGROUND OF THE INVENTION

1. Field of the Invention

The invention relates to ladder frame polyether conjugates and their use to transport molecules (cell permeable, cell impermeability/low permeability) across cell membranes/cell walls/organelle membranes/organelle walls in living organisms, tissues, cell cultures or membrane preparations (collectively, hereinafter “biological membranes”).

2. Description of the Related Art

There are many obstacles in developing treatments which allow the delivery of a compound across a biological membrane to its active site in a biological system. The delivery of an active agent has been assisted, primarily, through the use of certain peptides (see, e.g., U.S. Publication Application Nos.: 20060293242: “Transporting of Taxoid Derivatives through the Blood Brain Barrier”; 20080234197: “Method(s) of stabilizing and potentiating the actions and administration of brain-derived neurotrophic factor (BDNF)”; and 20090074857: “Glycerophospho-lipids for the improvement of cognitive functions”.

Ciguatoxins were identified as a new class of compounds, known as ladder frame polyethers, from an extract of predatory fish from the south pacific. Subsequently, many more polyether ladder compounds have been isolated from marine organisms. A ladder frame polyether compound is a synthetic, natural or semi-synthetic compound having two or more fused cyclic ether moieties. Examples of these compounds include, but are not limited to, brevetoxins, maitotoxins, yessotoxins, gambierols, hemibrevetoxins, brevenals, tamulamides, and brevisins. The ladder frame polyether compounds listed above is not intended to be exhaustive or limiting. Many of these compounds have unique biological activities such as acting on ion selective channels.

Examples of drugs that do not cross biological membranes very easily include many anticancer drugs (doxorubicin, paclitaxel, vincristine, and vinblastine), azidothimadine used to treat HIV, and neurotrophins (small polypeptides) to treat neurodegenerative disorders. Examples of compounds with low membrane permeability that are currently being used as biological tools could be enhanced by increasing their transport across membranes include, without limitation, charged fluorescent compounds, charged fluorescently labeled compounds, pH sensitive dyes, ion sensitive dyes, selective organelle stains, and antibodies.

### SUMMARY OF THE INVENTION

The inventors have discovered that ladder frame polyether compounds, when conjugated to membrane impermeable compounds such as large polar and/or ionic compounds and/or zwitterionic compounds, provide conjugates that are rapidly transported across the cell membranes and cell walls. This indicates that the ladder frame polyether compounds are useful in transporting other compounds across cell membranes and cell walls that would not otherwise be easily transported into cells.

The present invention is directed to conjugates of a ladder frame polyether compound (collectively, hereinafter, ‘escorter’) and at least one biologically active compound or research compound, and salts, solvates, hydrates or coordination compounds thereof. It is further directed to methods of delivering, without limitation, biologically active compounds, (for example, small molecule therapeutic drugs, nutraceuticals, hormones, proteins, peptides, amino acids) and research compounds (for example fluorescent markers, colorometric dyes, radioactive ligands) (collectively, hereinafter ‘active agents’) across biological membranes.

The invention comprehends conjugate molecules, and salts, solvates, hydrates, or coordination compounds thereof, wherein the escorter molecule portion facilitates entry of at least one active agent into cells and/or subcellular organelles. A linker component may be part of the conjugate to provide for retention of biological activity of the active agent, or allow release of the active agent, through one or more of various mechanisms, from the escorter.

The conjugates of the invention have numerous potential advantages. Firstly, the conjugates of the invention promote the intracellular entry of a variety of useful bioactive compounds and markers across biological membranes at pharmacokinetic rates. Secondly, the conjugates allow for transportation through the blood-brain barrier. Thirdly, the compositions of the invention incorporate various linkers that allow pharmacologically-relevant dosage rates of drug released from escorter-active agent molecules to be engineered into such compositions, thereby potentially increasing their biological efficacy, safety and usefulness.

### DETAILED DESCRIPTION

In an embodiment, the invention provides conjugates that may be represented by Formula I:

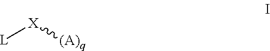

where L is a ladder frame compound;
X represents an optional linker;
each A is independently a biologically active compound or research compound; and
q represents an integer of from 1-5.

In Formula I, A is depicted as being covalently connected to L, optionally through linker X.

For clarity, where q is greater than 1, the manner of covalently connecting each A group to L is independent from the manner of connecting other A groups, and may be a direct covalent connection of A to L, or an indirect connection through a linker X. When more than one linker group X is employed, the linker groups are the same or different.

Particular conjugates of Formula I include those where q is 1, 2, or 3.

In one embodiment, the disclosure provides a conjugate comprising a ladder frame polyether compound and at least one of the groups consisting of biologically active compounds and research compounds, or a salt, solvate, hydrate or coordination compound thereof. In another embodiment, the at least one compound is a biologically active compound. In yet another embodiment, the at least one compound is a research compound.

In one embodiment, the disclosure provides a conjugate comprising a ladder frame polyether compound and the at least one biologically active compound, which is a drug or pro-drug. In another embodiment, the biologically active compound is a pesticide.

In one embodiment, the disclosure provides a conjugate comprising a ladder frame polyether compound and the at least one research compound, which is a fluorophore.

In certain embodiments, the disclosure as described above provides a conjugate wherein the ladder frame polyether compound is a brevisin compound.

In certain embodiments, the disclosure as described above provides a conjugate further comprises one or more linkers connecting one or more of A to L. In some embodiments, the at least one compound is a biologically active compound. In other embodiments, the at least one compound is a research compound.

In one embodiment, the disclosure provides a pharmaceutical formulation comprising a pharmaceutically effective amount of the conjugate of as described in the above embodiments and at least one pharmaceutically acceptable carrier.

In one embodiment, the disclosure provides a formulation for use on non-animal target species comprising an effective amount of the conjugate as described in the above embodiments and at least one adjuvant.

In one embodiment, the disclosure provides a formulation for the control of insects comprising an effective amount of the conjugate as described in the above embodiments and at least one adjuvant.

In one embodiment, the disclosure provides a method of improving the cellular uptake of a compound selected from the group consisting of one or more biologically active compound and research compound comprising administering the conjugate as described in the above embodiments to a target species. In one embodiment, the target species is an animal. In yet another embodiment, the target species is a plant. In yet another embodiment, the target species is fungus and yeast.

In one embodiment, the disclosure provides a method of treating a disease state in an animal in need of treatment comprising administering an effective amount of the conjugate as described in the above embodiments, or a pharmaceutically acceptable salt, solvate, hydrate or coordination compound thereof. In one embodiment, the escorter is a brevisin compound.

In one embodiment, the disclosure provides a method of treating a non-animal pest selected from the group consisting of an agricultural and horticultural pest, comprising applying the formulation for the control of insects comprising an effective amount of the conjugate as described in the above embodiments and at least one adjuvant. In one embodiment, the escorter is a brevisin compound.

In one embodiment, the disclosure provides a method of improving cellular uptake of a biologically active molecule or a research molecule comprising covalently coupling the molecule to a ladder frame polyether compound. In one embodiment, the coupling comprises creating a bond from the molecule to a linking group, and then creating a bond between the linking group and the ladder frame polyether compound.

In another embodiment, the coupling comprises creating a bond between the ladder frame polyether compound to a linking group, and then creating a bond between the linking group and the molecule.

In another embodiment, the invention provides methods of improving cellular uptake of biologically active molecules and research molecules. These methods comprise covalently coupling the molecule to a ladder frame polyether compound. In a particular embodiment, the coupling comprises creating a bond from the molecule to a linking group, and then creating a bond between the linking group and the ladder frame polyether compound. In an alternative embodiment, the coupling comprises creating a bond between the ladder frame polyether compound to a linking group, and then creating a bond between the linking group and the molecule.

In still another embodiment, the invention provides methods for determining the effect of a biologically active molecule or a research molecule on a target species. These methods comprise administering the biologically active molecule or the research molecule to the target species, as a conjugate, where the conjugate comprises the biologically active molecule or the research molecule covalently linked, optionally through a linker group, to a ladder frame polyether compound.

In yet another embodiment, the invention provides methods for determining the effect of a biologically active molecule or a research molecule on tissue or cells from a target species. These methods comprise contacting the biologically active molecule or the research molecule with the tissue or cells, where the conjugate comprises the biologically active molecule or the research molecule covalently linked, optionally through a linker group, to a ladder frame polyether compound.

In another embodiment, the invention provides a kit comprising a package containing a ladder frame polyether compound and labeling indicating that the ladder frame polyether compound is for use in an assay for determining the effect of a biologically active molecule or a research molecule on a target species, or on tissue or cells from a target species.

### DEFINITION OF TERMS

The term “drug” or “therapeutic agent” refers to an active agent or pro-drug that has a pharmacological activity or benefits health when administered in a therapeutically effective amount. Examples of such agents include, without limitation, naturally occurring biological agents (e.g., enzymes, proteins, polynucleotides, antibodies, polypeptides) and synthetic and semi-synthetic compounds.

A “marker”, “label” or a “detectable moiety” is a compound detectable by spectroscopic, photochemical, biochemical, immunochemical, chemical, or other physical means. For example, labels suitable for use in the present invention include, for example, radioactive labels (e.g., ³²P), fluorophores (e.g., fluorescein), electron dense reagents, enzymes (e.g., as commonly used in an ELISA), biotin, digoxigenin, or haptens and proteins which can be made detectable, e.g., by incorporating a radiolabel into the hapten or peptide, or used to detect antibodies specifically reactive with the hapten or peptide.

A “therapeutically effective amount” is defined as an amount of one or more biologically active compounds required to reduce or lessen at least one symptom of the disease being treated or to reduce or delay onset of one or more clinical markers, symptoms of the disease or the disease itself.

“Detecting” refers to determining the presence, absence, or amount of an analyte in a sample, and can include quantifying the amount of the analyte in a sample or per cell in a sample.

“Escorter” refers to a ladder frame polyether compound capable of forming the conjugates of the invention.

“Linker” refers to a moiety, e.g., an atom or group of atoms (molecular fragment), that joins two other molecules, through covalent, ionic, van der Waals or hydrogen bonds. Specifically, the term “linker” refers to a group or groups that (1) covalently links the escorter to the biologically active compounds or research compounds or both and/or (2) covalently links escorter one to another and to the biologically active compounds or research compounds or both; non-limiting illustrations of the latter include escorter-linker-escorter, escorter-linker-escorter-linker-research compound, and the like. Within any particular conjugate, the linker connecting escorter together or the escorted and the biologically active compounds or research compounds may be the same or different (i.e., may have the same or different chemical structures).

“Pesticide” refers to “any substance (or mixture of substances) intended for a pesticidal purpose, i.e. use for the preventing, destroying, repelling, or mitigating any pest or use as a plant regulator, defoliant or desiccant” (40 C.F.R. 152.15) and includes, without limitation, herbicides, fungicides and insecticides as used in agriculture or horticulture.

As used in the specification and appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

As used herein, the terms “treatment” and “treating” encompass prophylactic administration of at least one conjugate or pharmaceutical composition comprising such conjugate(s) (“prophylaxis”) as well as remedial therapy to reduce or eliminate a targeted disease or disorder. Prophylactic administration is intended for preventing disorders or preventing recurrence of disorders and may be used to treat a subject that is at risk of having or suffering from one or more targeted disorders. Thus, as used herein, the term “treatment”, or a derivative thereof, contemplates partial or complete inhibition of the targeted disease state, when at least one active ingredient of the invention is administered prophylactically or following the onset of the disease state for which such active ingredient(s) is/are administered.

As used herein, the term “subject” encompasses animals, fungi, bacteria, single cell organisms, and the like.

As used herein, the term “animal” includes, vertebrates and invertebrates, such as, without limitation, mammals (including humans), fish, reptiles, amphibians, birds, worms, arthropods, mollusks, and the like.

As used herein, “target species” encompasses, for example and without limitation, animals, plants (including crops, weeds and the like), insects, fungi, yeast, bacteria, algae, single cell organisms, and the like. Unless defined otherwise, all scientific and technical terms used herein have the same meaning as commonly understood by one of skill in the art to which this invention belongs.

All patents and publications referred to herein are hereby incorporated by reference for all purposes.

Ladder Frame Polyether Carrier Molecule (Escorter):

Ladder frame polyether carrier molecules, frequently from marine microorganisms, include but are not limited to brevenal (see, e.g., U.S. Pat. No. 7,202,271; Takamura, H. et al, Org. Lett., 2009, 11 (12), 2531-2354), brevisin (see, e.g., Satake M, et al, J. Org. Chem. 2009, 74, 989-994; Van Wagoner, R., et al, J. Nat. Prod., 2010, 73 (6), 1177-1179; Karangu, T., et al, Tet. Lett., 2010, 51 (35), 4673-4676), tamulamide (see, e.g., Bourdelais, A., et al, J. Nat. Prod. 2010, 73, 536-540), BTX-B5, PbTx-2, PbTx-3 and other brevetoxins (see, e.g., U.S. Publication Application No. 20070111243) and metabolites and congeners thereof, hemibrevetoxins (see, e.g., Prasad A V K, Shimizu Y., J. Am. Chem. Soc. 1989; 111:6476-6477), gambierols and gambieric acids (see, e.g., Kodata, I, et al, J. Am. Chem. Soc. 2003, 125, 46-47), gymnosins, ciguatoxins, and yessotoxins (see, e.g., Murata, M, et al, Tetrahedron Lett. 1987; 28, 5869-5872), semi-synthetic derivatives of the preceeding compounds and synthetically derived polyethers. Ladder frame polyether compounds have the following generic structural fragment:

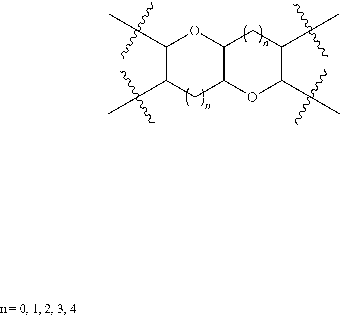

Any of the carbon atoms in the above fragment, including the bridgehead carbon atoms, may be substituted with non-hydrogen groups. Representative groups include C₁-C₆alkyl, C₂-C₆ alkenyl, hydroxy(C₁-C₆)alkyl, amino(C₁-C₆)alkyl, halo(C₁-C₆)alkyl, C₃-C₇cycloalkyl(C₁-C₆)alkyl, and C₁-C₆alkoxy(C₁-C₆)alkyl. Further, the polyether, polycyclic rings may contain one or more sites of unsaturation, and are optionally substituted as set forth herein.

Examples of escorters include those having the Brevetoxin A backbone:

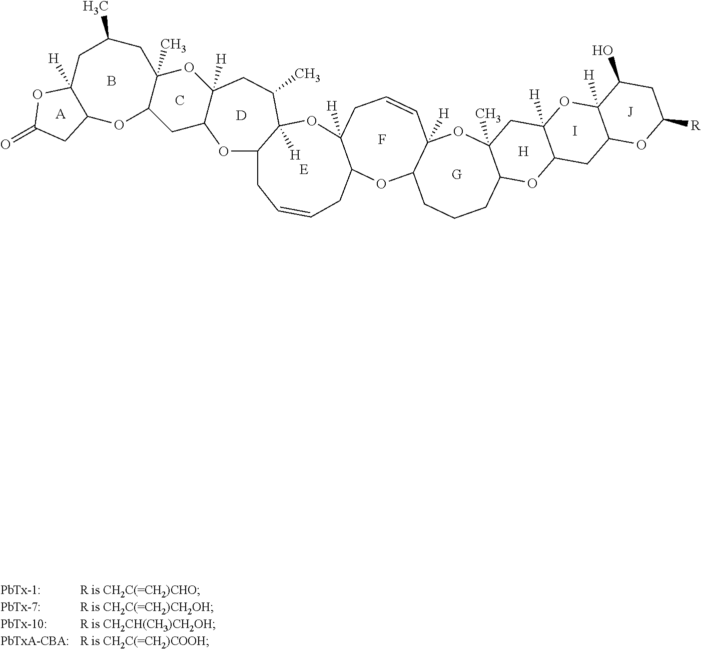

Other examples of escorters have the Brevetoxin B backbone:

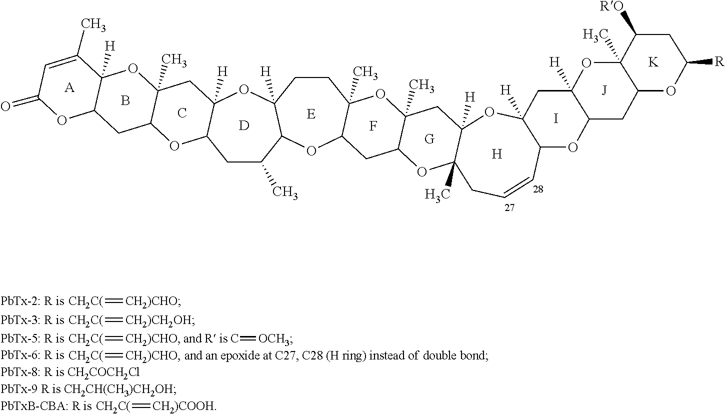

Other examples of escorter molecules include, for example, those derived from brevenal, hemibrevetoxin B, tamulamide, brevisin, gambieric acids, naturally derived ciguatoxins and their derivatives:

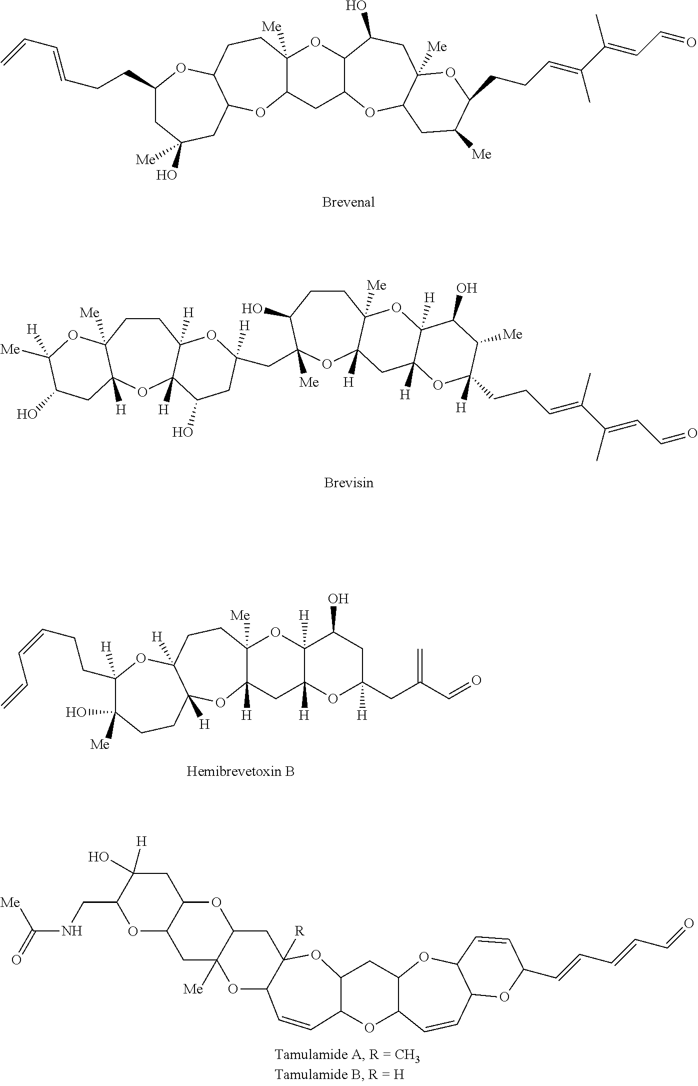

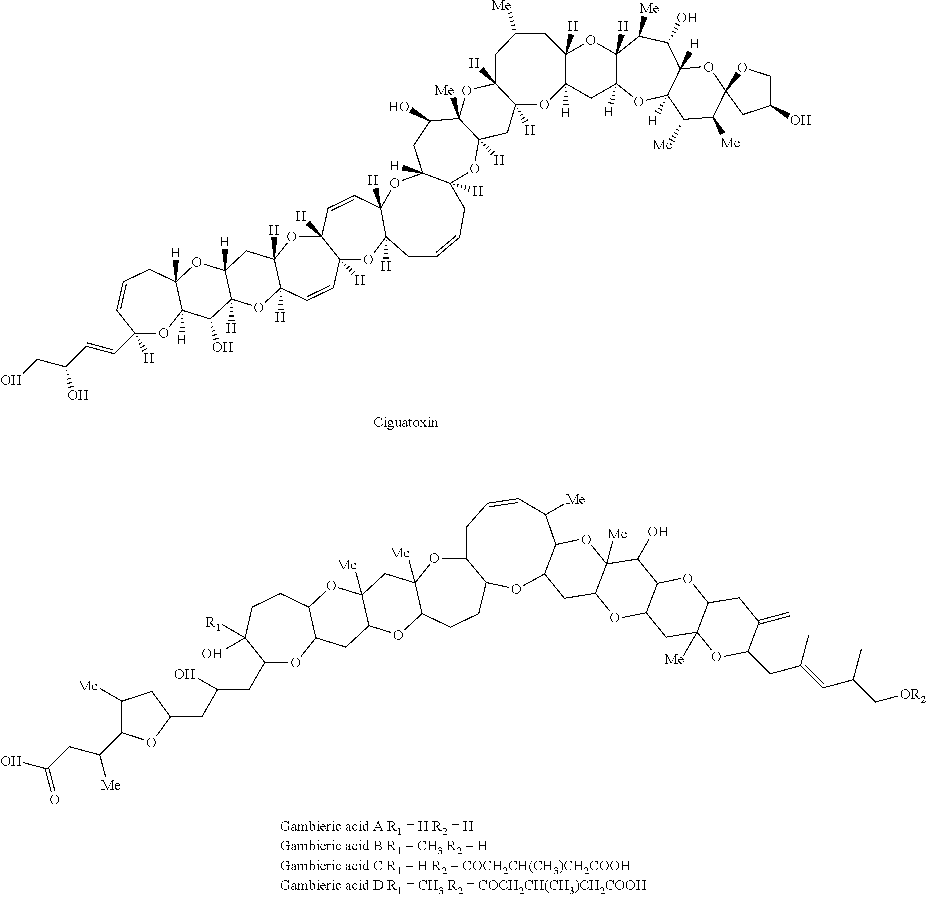

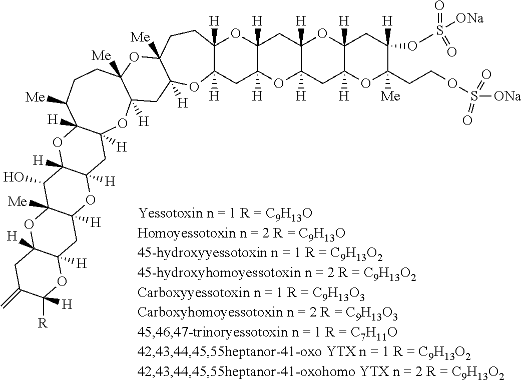

Other embodiments of the escorter include modifications of a given escorter such that the molecule includes a linker moiety, wherein the linker moiety has a functional group that can form a covalent bond with at least one active agent.

A further embodiment is the use of a labile linker between the escorter and the active agent of interest. In this embodiment, the escorter would be covalently attached through a non-labile bond to the linker. The active agent would be covalently attached to the linker through a labile bond. Arrangement of the functionalities would then allow escorter-facilitated transport through a biological membrane and then release of the active agent. These releasing events include, but are not limited to, photolytic events, enzymatic action and changes in pH.

In specific embodiments, linker moieties include, for example and without limitation, imines, acetals, ketals, thioacetals, thioketals, esters, ethers, amines, amides, carbonates, carbamates, hydrazones, acyl hydrazones, aminols, sulfonyl hydrazones, hydrazides, diacyl hydrazides, acyl alkylidene hydrazides, phosphates, and thioalkylamides.

Biologically Active Compounds:

Biologically active compounds, according to the invention, include agents that can affect a biological process. Such compounds for use in the compositions and methods of the invention are, for example and without limitation, therapeutic agents, including drugs, pro-drugs and diagnostic agents, insecticides, fungicides, growth hormones, nutrients and herbicides, and formulations thereof.

In other embodiments, biologically active compounds are conjugated with a substituent on the respective escorter.

Examples of drugs or therapeutic agents include those substances that are used in the prevention, diagnosis, alleviation, treatment or cure of a disease or condition or for use as a biological tool. It is particularly contemplated that the agent is not an agent that causes a disease.

Generally, biologically active compounds to be conjugated with one or more escorters do not appear to be limited in size.

Examples of biologically active compounds those used, for example and without limitation, as follows an anti-inflammatory drug substance, a urinary tract analgesic, an anti-angina drug, an antihelminthic, an anti-arrhythmic agent, an anti-asthma drug, an anti-bacterial drug, an anti-cancer drug substance, an immunosuppressant, an anti-coagulant drug substance, an anti-diabetic drug substance, an anti-epileptic, an anti-fungal, an anti-gout drug substance, an antihistamine, an allergy medication, an antihypertensive, an anti-malarial, a headache treatment drug substance, an anti-migraine agent, an anti-muscarinic drug substance, an anti-protozoal drug substance, an anti-thyroid drug, anti-tussive, an antiviral drug substance, an anxiolytic, a sedatives, a hypnotic, an appetite suppressant, an anti-obesity drug, an eating disorder treatment drug substance, a cardiovascular drug substance, a corticosteroid, an erectile dysfunction drug substance, a gastrointestinal drug substance, genetic material, a keratolytic, a lipid-regulating drug substance, a muscle relaxant, a neurodegenerative treatment agent, a nitrate and other anti-anginal drug substance, a neuroleptic drug substance, a nutritional agent, an opioid analgesic, a peptidyl drug substance, a sex hormone, an androgenic drug substance.

Suitable active agents include, but are not limited to, psychopharmacological agents, such as (1) central nervous system depressants, e.g., general anesthetics (barbiturates, benzodiazepines, steroids, cyclohexanone derivatives, and miscellaneous agents), sedative-hypnotics (benzodiazepines, barbiturates, piperidinediones and triones, quinazoline derivatives, carbamates, aldehydes and derivatives, amides, acyclic ureides, benzazepines and related drugs, phenothiazines, etc.), central voluntary muscle tone modifying drugs (anticonvulsants, such as hydantoins, barbiturates, oxazolidinediones, succinimides, acylureides, glutarimides, benzodiazepines, secondary and tertiary alcohols, dibenzazepine derivatives, valproic acid and derivatives, GABA analogs, etc.), analgesics (morphine and derivatives, oripavine derivatives, morphinan derivatives, phenylpiperidines, 2,6-methane-3-benzazocaine derivatives, diphenylpropylamines and isosteres, salicylates, p-aminophenol derivatives, 5-pyrazolone derivatives, arylacetic acid derivatives, fenamates and isosteres, etc.) and antiemetics (anticholinergics, antihistamines, antidopaminergics, etc.), (2) central nervous system stimulants, e.g., analeptics (respiratory stimulants, convulsant stimulants, psychomotor stimulants), narcotic antagonists (morphine derivatives, oripavine derivatives, 2,6-methane-3-benzoxacine derivatives, morphinan derivatives) nootropics, (3) psychopharmacologicals, e.g., anxiolytic sedatives (benzodiazepines, propanediol carbamates) antipsychotics (phenothiazine derivatives, thioxanthine derivatives, other tricyclic compounds, butyrophenone derivatives and isosteres, diphenylbutylamine derivatives, substituted benzamides, arylpiperazine derivatives, indole derivatives, etc.), antidepressants (tricyclic compounds, MAO inhibitors, etc.), (4) respiratory tract drugs, e.g., central antitussives (opium alkaloids and their derivatives); pharmacodynamic agents, such as (1) peripheral nervous system drugs, e.g., local anesthetics (ester derivatives, amide derivatives), (2) drugs acting at synaptic or neuroeffector junctional sites, e.g., cholinergic agents, cholinergic blocking agents, neuromuscular blocking agents, adrenergic agents, antiadrenergic agents, (3) smooth muscle active drugs, e.g., spasmolytics (anticholinergics, musculotropic spasmolytics), vasodilators, smooth muscle stimulants, (4) histamines and antihistamines, e.g., histamine and derivative thereof (betazole), antihistamines (H1-antagonists, H2-antagonists), histamine metabolism drugs, (5) cardiovascular drugs, e.g., cardiotonics (plant extracts, butenolides, pentadienolids, alkaloids from erythrophleum species, ionophores, adrenoceptor stimulants, etc), antiarrhythmic drugs, antihypertensive agents, antilipidemic agents (clofibric acid derivatives, nicotinic acid derivatives, hormones and analogs, antibiotics, salicylic acid and derivatives), antivaricose drugs, hemostyptics, (6) blood and hemopoietic system drugs, e.g., antianemia drugs, blood coagulation drugs (hemostatics, anticoagulants, antithrombotics, thrombolytics, blood proteins and their fractions), (7) gastrointestinal tract drugs, e.g., digestants (stomachics, choleretics), antiulcer drugs, antidiarrheal agents, (8) locally acting drugs; chemotherapeutic agents, such as (1) anti-infective agents, e.g., ectoparasiticides (chlorinated hydrocarbons, pyrethins, sulfurated compounds), anthelmintics, antiprotozoal agents, antimalarial agents, antiamebic agents, antileiscmanial drugs, antitrichomonal agents, antitrypanosomal agents, sulfonamides, antimycobacterial drugs, antiviral chemotherapeutics, etc., and (2) cytostatics, i.e., antineoplastic agents or cytotoxic drugs, such as alkylating agents, e.g., Mechlorethamine hydrochloride (Nitrogen Mustard, Mustargen, HN2), Cyclophosphamide (Cytovan, Endoxana), Ifosfamide (IFEX), Chlorambucil (Leukeran), Melphalan (Phenylalanine Mustard, L-sarcolysin, Alkeran, L-PAM), Busulfan (Myleran), Thiotepa (Triethylenethiophosphoramide), Carmustine (BiCNU, BCNU), Lomustine (CeeNU, CCNU), Streptozocin (Zanosar) and the like; plant alkaloids, e.g., Vincristine (Oncovin), Vinblastine (Velban, Velbe), Paclitaxel (Taxol), and the like; antimetabolites, e.g., Methotrexate (MTX), Mercaptopurine (Purinethol, 6-MP), Thioguanine (6-TG), Fluorouracil (5-FU), Cytarabine (Cytosar-U, Ara-C), Azacitidine (Mylosar, 5-AZA) and the like; antibiotics, e.g., Dactinomycin (Actinomycin D, Cosmegen), Doxorubicin (Adriamycin), Daunorubicin (duanomycin, Cerubidine), Idarubicin (Idamycin), Bleomycin (Blenoxane), Picamycin (Mithramycin, Mithracin), Mitomycin (Mutamycin) and the like, and other anticellular proliferative agents, e.g., Hydroxyurea (Hydrea), Procarbazine (Mutalane), Dacarbazine (DTIC-Dome), Cisplatin (Platinol) Carboplatin (Paraplatin), Asparaginase (Elspar) Etoposide (VePesid, VP-16-213), Amsarcrine (AMSA, m-AMSA), Mitotane (Lysodren), Mitoxantrone (Novatrone), and the like. Chemotherapeutic agents are those, which in the free form, demonstrate unacceptable systemic toxicity at desired doses. Particularly are cardiotoxic compounds that are useful therapeutics but are dose limited by cardiotoxicity. A classic example is adriamycin (also known as doxorubicin) and its analogs, such as daunorubicin.

In yet another embodiment, the active agent is a stimulant, and a drug substance for treatment of narcolepsy, attention deficit disorder (ADD) and attention deficit hyperactivity disorder (ADHD).

In some embodiments, the active agent is an anti-inflammatory drug substances and non-opioid analgesics including, for example and without limitation, aloxiprin, auranofin, azapropazone, azathioprine, benorylate, butorphenol, capsaicin, celecoxib, diclofenac, diflunisal, esonarimod, etodolac, fenbufen, fenoprofen calcium, flurbiprofen, ibuprofen, indomethacin, ketoprofen, ketorolac, leflunomide, meclofenamic acid, mefenamic acid, nabumetone, naproxen, novantrone, oxaprozin, oxyphenbutazone, parecoxib, phenylbutazone, piclamilast, piroxicam, rofecoxib, ropivacaine, sulindac, tetrahydrocannabinol, tramadol, tromethamine, valdecoxib, and ziconotide, as well as the urinary analgesics phenazopyridine and tolterodine.

In other embodiments, the active agent is an anti-angina drug substances including, for example and without limitation, mibefradil, refludan, nahnefene, carvedilol, cromafiban, lamifiban, fasudil, ranolazine, tedisamil, nisoldipine, and tizanidine.

In still other embodiments, the active agent is an anthelmintics including, for example and without limitation, albendazole, cambendazole, ivermectin, mebendazole, oxamniquine, oxfendazole, oxantel embonate, praziquantel, pyrantel embonate and thiabendazole. Suitable active agents include, but are not limited to anthemintics such as arecoline, aspidin, aspidinol, dichlorophene, embelin, kosin, napthalene, niclosamide, pelletierine, quinacrine, alantolactone, amocarzine, amoscanate, ascaridole, bephenium, bitoscanate, carbon tetrachloride, carvacrol, cyclobendazole, diethylcarbamazine, etc.

In yet another embodiment, the active agent is an anti-arrhythmic agents including, for example and without limitation, such as amiodarone, disopyramide, flecainide acetate and quinidine sulfate.

In some embodiments, the active agent is an anti-asthma drug substances including, for example and without limitation, zileuton, zafirlukast, terbutaline sulfate, montelukast, and albuterol.

In some embodiments, the active agent is an anti-bacterial drug substances including, for example and without limitation, alatrofloxacin, azithromycin, baclofen, benethamine penicillin, cinoxacin, ciprofloxacin, clofazimine, cloxacillin, demeclocycline, dirithromycin, doxycycline, ethionamide, furazolidone, grepafloxacin, imipenem, levofloxacin, lorefloxacin, moxifloxacin, nalidixic acid, nitrofurantoin, norfloxacin, ofloxacin, rifampicin, rifabutine, rifapentine, sparfloxacin, spiramycin, sulphabenzamide, sulphadoxine, sulphamerazine, sulphacetamide, sulphadiazine, sulphafurazole, sulphamethoxazole, sulphapyridine, tetracycline, trimethoprim, trovafloxacin, and vancomycin. Suitable active agents include, but are not limited to: Antibiotics, such as: aminoglycosides, e.g., amikacin, apramycin, arbekacin, bambermycins, butirosin, dibekacin, dihydrostreptomycin, fortimicin, gentamicin, isepamicin, kanamycin, micronomcin, neomycin, netilmicin, paromycin, ribostamycin, sisomicin, spectinomycin, streptomycin, tobramycin, trospectomycin; amphenicols, e.g., azidamfenicol, chloramphenicol, florfenicol, and theimaphenicol; ansamycins, e.g., rifamide, rifampin, rifamycin, rifapentine, rifaximin; beta.-lactams, e.g., carbacephems, carbapenems, cephalosporins, cehpamycins, monobactams, oxaphems, penicillins; lincosamides, e.g., clindamycin, lincomycin; macrolides, e.g., clarithromycin, dirithromycin, erythromycin, etc.; polypeptides, e.g., amphomycin, bacitracin, capreomycin, etc.; tetracyclines, e.g., apicycline, chlortetracycline, clomocycline, etc.; synthetic antibacterial agents, such as 2,4-diaminopyrimidines, nitrofurans, quinolones and analogs thereof, sulfonamides, and sulfones.

In still other embodiments, the active agent is an anti-cancer drug substance and immunosuppressant including, for example and without limitation, alitretinoin, aminoglutethimide, amsacrine, anastrozole, azathioprine, bexarotene, bicalutamide, biricodar, bisantrene, busulfan, camptothecin, candoxatril, capecitabine, cytarabine, chlorambucil, cyclosporin, dacarbazine, decitabine, ellipticine, estramustine, etoposide, gemcitabine, irinotecan, lasofoxifene, letrozole, lomustine, melphalan, mercaptopurine, methotrexate, mitomycin, mitotane, mitoxantrone, mofetil, mycophenolate, nebivolol, nilutamide, paclitaxel, palonosetron, procarbazine, ramipril, rubitecan, sirolimus, tacrolimus, tamoxifen, teniposide, testolactone, thalidomide, tirapazamine, topotecan, toremifene citrate, vitamin A, vitamin A derivatives, and zacopride.

In yet another embodiment, the active agents is an anti-coagulant and other drug substance for preventing and treating stroke including, for example and without limitation, cilostazol, citicoline, clopidogrel, cromafiban, dexanabinol, dicumarol, dipyridamole, nicoumalone, oprelvekin, perindopril erbumine, phenindione, ramipril, repinotan, ticlopidine, tirofiban, and heparin, including heparin salts formed with organic or inorganic bases, and low molecular weight heparin, i.e., heparin fragments generally having a weight average molecular weight in the range of about 1000 to about 10,000 D and exemplified by enoxaparin, dalteparin, danaproid, gammaparin, nadroparin, ardeparin, tinzaparin, certoparin, and reviparin.

In some embodiments, the active agent is an anti-diabetic drug substance include, for example and without limitation, acetohexamide, chlorpropamide, ciglitazone, farglitazar, glibenclamide, gliclazide, glipizide, glucagon, glyburide, glymepiride, miglitol, nateglinide, pimagedine, pioglitazone, repaglinide, rosiglitazone, tolazamide, tolbutamide, triampterine, troglitazone and voglibose.

In still other embodiments, the active agent is an anti-epileptic including, for example and without limitation, beclamide, carbamazepine, clonazepam, ethotoin, felbamate, fosphenyloin, lamotrigine, methoin, methsuximide, methylphenobarbitone, oxcarbazepine, paramethadione, phenacemide, phenobarbitone, phenyloin, phensuximide, primidone, sulthiame, tiagabine, topiramate, valproic acid, and vigabatrin.

In yet another embodiment, the active agent is an anti-fungal drug substance including, for example and without limitation, butenafine, clotrimazole, econazole nitrate, flucytosine, griseofulvin, itraconazole, ketoconazole, miconazole, natamycin, sulconazole nitrate, oxiconazole, terbinafine, tioconazole and undecenoic acid. Suitable active agents also include, but are not limited to antifungal agents, such as: polyenes, e.g., amphotericin B, candicidin, dermostatin, filipin, fungichromin, hachimycin, hamycin, lucensomycin, mepartricin, natamycin, nystatin, pecilocin, perimycin; synthetic antifungals, such as allylamines, e.g., butenafine, naftifine, terbinafine; imidazoles, e.g., bifonazole, butoconazole, chlordantoin, chlormidazole, etc., thiocarbamates, e.g., tolciclate, triazoles, e.g., fluconazole, itraconazole, terconazole.

In some embodiments, the active agent is an anti-gout drug substance including, for example and without limitation, allopurinol, probenecid and sulphin-pyrazone.

In other embodiments, the active agent is an antihistamine and allergy medication including, for example and without limitation, acrivastine, astemizole, chlorpheniramine, cinnarizine, cetirizine, clemastine, cyclizine, cyproheptadine, desloratadine, dexchlorpheniramine, dimenhydrinate, diphenhydramine, epinastine, fexofenadine, flunarizine, loratadine, meclizine, mizolastine, oxatomide, and terfenadine.

In still other embodiments, the active agent is an antihypertensive drug substance include, for example and without limitation, amlodipine, benazepril, benidipine, candesartan, captopril, carvedilol, darodipine, dilitazem, diazoxide, doxazosin, enalapril, epleronone, eposartan, felodipine, fenoldopam, fosinopril, guanabenz, iloprost, irbesartan, isradipine, lercardinipine, lisinopril, losartan, minoxidil, nebivolol, nicardipine, nifedipine, nimodipine, nisoldipine, omapatrilat, phenoxybenzamine, prazosin, quinapril, reserpine, semotiadil, sitaxsentan, terazosin, telmisartan, and valsartan.

In yet other embodiments, the active agent is an anti-malarial including, for example and without limitation, acedapsone, amodiaquin, arteether, artemether, artemisinin, artesunate, atovaquone, bebeerine, berberine, chirata, chlorguanide, chloroquine, chlorprogaunil, cinchona, cinchonidine, cinchonine, cycloguanil, gentiopicrin, halofantrine, hydroxychloroquine, mefloquine hydrochloride, 3-methylarsacetin, pamaquine, plasmocid, primaquine, proguanil, pyrimethamine, quinacrine, quinidine, quinine, quinocide, quinoline, dibasic sodium arsenate.

In some embodiments, the active agent is a drug substance for treating headaches, including anti-migraine agents including, for example and without limitation, almotriptan, butorphanol, dihydroergotamine, dihydroergotamine mesylate, eletriptan, ergotamine, frovatriptan, methysergide, naratriptan, pizotyline, rizatriptan, sumatriptan, tonaberstat, and zolmitriptan.

In other embodiments, the active agent is an anti-muscarinic drug substance including, for example and without limitation, atropine, benzhexyl, biperiden, ethopropazine, hyoscyamine, mepenzolate bromide, oxyphencyclimine, scopolamine, and tropicamide.

In still other embodiments, the active agent is an anti-protozoal drug substance including, for example and without limitation, atovaquone, clioquinol, decoquinate, diiodohydroxyquinoline, diloxanide furoate, dinitolmide, furazolidone, metronidazole, nimorazole, nitrofirazone, ornidazole and tinidazole. Suitable active agents also include, but are not limited to antiprotozoan agents such as: acranil, tinidazole, ipronidazole, ethylstibamine, pentamidine, acetarsone, aminitrozole, anisomycin, nifuratel, benzidazole, suramin, and the like.

In yet other embodiments, the active agent is an anti-thyroid drug substance including, for example and without limitation, carbimazole, paricalcitol, and propylthiouracil.

In some embodiments, the active agent is an anti-tussive including, for example and without limitation, benzonatate.

In other embodiments, the active agent is an antiviral drug substances including, for example and without limitation, antiherpes agents acyclovir, famciclovir, foscarnet, ganciclovir, idoxuridine, sorivudine, trifluridine, valacyclovir, and vidarabine, and other antiviral agents such as abacavir, amantadine, amprenavir, delviridine, didanosine, efavirenz, indinavir, interferon alpha, lamivudine, nelfinavir, nevirapine, ribavirin, rimantadine, ritonavir, saquinavir, stavudine, tipranavir, valganciclovir, zalcitabine, and zidovudine; and other antiviral agents such as abacavir, indinavir, interferon alpha, nelfinavir, ribavirin, rimantadine, tipranavir, ursodeoxycholic acid, and valganciclovir.

In still other embodiments, the active agent is an anxiolytic, sedative, and hypnotic including, for example and without limitation, alprazolam, amylobarbitone, barbitone, bentazepam, bromazepam, bromperidol, brotizolam, butobarbitone, carbromal, chlordiazepoxide, chlormethiazole, chlorpromazine, chlorprothixene, clonazepam, clobazam, clotiazepam, clozapine, dexmethylphenidate (d-threo-methylphenidate) diazepam, droperidol, ethinamate, flunanisone, flunitrazepam, triflupromazine, flupenthixol decanoate, fluphenazine, flurazepam, gabapentin, gaboxadol, .gamma.-hydroxybutyrate, haloperidol, lamotrigine, lorazepam, lormetazepam, medazepam, meprobamate, mesoridazine, methaqualone, methylphenidate, midazolam, modafinil, molindone, nitrazepam, olanzapine, oxazepam, pentobarbitone, perphenazine pimozide, pregabalin, prochlorperazine, pseudoephedrine, quetiapine, rispiridone, sertindole, siramesine, sulpiride, sunepitron, temazepam, thioridazine, triazolam, zaleplon, zolpidem, and zopiclone.

In yet other embodiments, the active agent is an appetite suppressant, anti-obesity drug substance and drug substance for treatment of eating disorders including, for example and without limitation, amphetamine, bromocriptine, dextroamphetamine, diethylpropion, lintitript, mazindol, methamphetamine, orlistat, phentermine, and topiramate.

In some embodiments, the active agent is a cardiovascular drug substance including, for example and without limitation, angiotensin converting enzyme (ACE) inhibitors such as enalapril, ramipril, perindopril erbumine, 1-carboxymethyl-3-1-carboxy-3-phenyl-(1S)-propylamino-2,3,4,5-tetrahydro-1H-(3S)-1-benzazepine-2-one, 3-(5-amino-1-carboxy-1S-pentyl)amino-2,3,4,5-tetrahydro-2-oxo-3S-1H-1-benzazepine-1-acetic acid or 3-(1-ethoxycarbonyl-3-phenyl-(1S)-propylamino)-2,3,4,5-tetrahydro-2-oxo-(3S)-benzazepine acid monohydrochloride.

In another embodiment, the active agent is a cardiac glycosides and cardiac inotropes such as aminone, digoxin, digitoxin, enoximone, lanatoside C, medigoxin, and milrinone.

In still another embodiment, the active agent is a calcium channel blockers such as verapamil, nifedipine, nicardipene, felodipine, isradipine, nimodipine, amlodipine and diltiazem.

In other embodiments, the active agent is a beta-blockers such as acebutolol, alprenolol, atenolol, labetalol, metoprolol, nadolol, oxyprenolol, pindolol, propafenone, propranolol, esmolol, sotalol, timolol, and acebutolol.

In still another embodiment, the active agent is an antiarrhythmic such as moricizine, dofetilide, ibutilide, nesiritide, procainamide, quinidine, disopyramide, lidocaine, phenyloin, tocamide, mexiletine, flecamide, encamide, bretylium and amiodarone.

In yet another embodiment, the active agent is a cardioprotective agent such as dexrazoxane and leucovorin.

In another embodiment, the active agent is a vasodilator such as nitroglycerin.

In yet another embodiment, the active agent is a diuretic agent such as azetazolamide, amiloride, bendroflumethiazide, bumetanide, chlorothiazide, chlorthalidone, ethacrynic acid, furosemide, hydrochlorothiazide, metolazone, nesiritide, spironolactone, and triamterine.

In other embodiments, the active agent is a miscellaneous cardiovascular drugs such as monteplase and corlopam.

In other embodiments, the active agent is a corticosteroid including, for example and without limitation, beclomethasone, betamethasone, budesonide, cortisone, desoxymethasone, dexamethasone, fludrocortisone, flunisolide, fluocortolone, fluticasone propionate, hydrocortisone, methylprednisolone, prednisolone, prednisone and triamcinolone.

In still other embodiments, the active agent is an erectile dysfunction drug substance including, for example and without limitation, pomorphine, phentolamine, and vardenafil.

In yet other embodiments, the active agent is a gastrointestinal drug substance including, for example and without limitation, alosetron, bisacodyl, cilansetron, cimetidine, cisapride, diphenoxylate, domperidone, esomeprazole, famotidine, granisetron, lansoprazole, loperamide, mesalazine, nizatidine, omeprazole, ondansetron, prantoprazole, rabeprazole sodium, ranitidine, risperidone, sulphasalazine, and tegaserod.

In some embodiments, the active agent is a genetic material including, for example and without limitation, nucleic acids, RNA, DNA, recombinant RNA, recombinant DNA, antisense RNA, antisense DNA, ribozymes, ribooligonucleotides, deoxyribonucleotides, antisense ribooligonucleotides, and antisense deoxyribooligonucleotides. Representative genes include those encoding for vascular endothelial growth factor, fibroblast growth factor, Bcl-2, cystic fibrosis transmembrane regulator, nerve growth factor, human growth factor, erythropoietin, tumor necrosis factor, and interleukin-2, as well as histocompatibility genes such as HLA-B7.

In other embodiments, the active agent is a keratolytic including, for example and without limitation, acetretin, calcipotriene, calcifediol, calcitriol, cholecalciferol, ergocalciferol, etretinate, retinoids, targretin, and tazarotene.

In still other embodiments, the active agent is a lipid-regulating drug substances that are generally classified as hydrophobic include HMG CoA reductase inhibitors including, for example and without limitation, atorvastatin, simvastatin, fluvastatin, pravastatin, lovastatin, cerivastatin, rosuvastatin, and pitavastatin, as well as other lipid-lowering (“antihyperlipidemic”) drug substances such as bezafibrate, beclobrate, binifibrate, ciprofibrate, clinofibrate, clofibrate, clofibric acid, ezetimibe, etofibrate, fenofibrate, fenofibric acid, gemfibrozil, nicofibrate, pirifibrate, probucol, ronifibrate, simfibrate, and theofibrate.

In yet other embodiments, the active agent is a muscle relaxant including, for example and without limitation, cyclobenzaprine, dantrolene sodium and tizanidine HCl.

In some embodiments, the active agent is an agent to treat neurodegenerative diseases, including active drug substances for treating Alzheimer's disease including, for example and without limitation, akatinol, donezepil, donepezil hydrochloride, dronabinol, galantamine, neotrofin, rasagiline, physostigmine, physostigmine salicylate, propentoffyline, quetiapine, rivastigmine, tacrine, tacrine hydrochloride, thalidomide, and xaliproden.

In other embodiments, the active agent is a drug substance for treating Huntington's Disease including, for example and without limitation, fluoxetine and carbamazepine.

In yet another embodiment, the active agent is an anti-parkinsonism drug useful such as, without limitation amantadine, apomorphine, bromocriptine, entacapone, levodopa (particularly a levodopa/carbidopa combination), lysuride, pergolide, pramipexole, rasagiline, riluzole, ropinirole, selegiline, sumanirole, tolcapone, trihexyphenidyl, and trihexyphenidyl hydrochloride.

In yet other embodiments, the active agent is a drug substance for treating ALS such as, without limitation, the anti-spastic agents baclofen, diazemine, and tizanidine.

In other embodiments, the active agent is a nitrate and other anti-anginal drug substances including, for example and without limitation, amyl nitrate, glyceryl trinitrate, isosorbide dinitrate, isosorbide mononitrate and pentaerythritol tetranitrate.

In still other embodiments, the active agent is a neuroleptic drug substance including, for example, antidepressant drugs, antimanic drugs, and antipsychotic agents, wherein antidepressant drugs include, without limitation, (a) the tricyclic antidepressants such as amoxapine, amitriptyline, clomipramine, desipramine, doxepin, imipramine, maprotiline, nortriptyline, protriptyline, and trimipramine, (b) the serotonin reuptake inhibitors such as citalopram, fluoxetine, fluvoxamine, paroxetine, sertraline, and venlafaxine, (c) monoamine oxidase inhibitors such as phenelzine, tranylcypromine, and (−)-selegiline, and (d) other antidepressants such as aprepitant, bupropion, duloxetine, gepirone, igmesine, lamotrigine, maprotiline, mianserin, mirtazapine, nefazodone, rabalzotan, sunepitron, trazodone and venlafaxine, and wherein antimanic and antipsychotic agents include, for example and without limitation, (a) phenothiazines such as acetophenazine, acetophenazine maleate, chlorpromazine, chlorpromazine hydrochloride, fluphenazine, fluphenazine hydrochloride, fluphenazine enanthate, fluphenazine decanoate, mesoridazine, mesoridazine besylate, perphenazine, thioridazine, thioridazine hydrochloride, trifluoperazine, and trifluoperazine hydrochloride, (b) thioxanthenes such as chlorprothixene, thiothixene, and thiothixene hydrochloride, and (c) other heterocyclic drugs such as carbamazepine, clozapine, droperidol, haloperidol, haloperidol decanoate, loxapine succinate, molindone, molindone hydrochloride, olanzapine, pimozide, quetiapine, risperidone, and sertindole.

In yet other embodiments, the active agent is a nutritional agent including, for example and without limitation, calcitriol, carotenes, dihydrotachysterol, essential fatty acids, non-essential fatty acids, phytonadiol, vitamin A, vitamin B.sub.2, vitamin D, vitamin E and vitamin K.

In some embodiments, the active agent is an opioid analgesic including, for example and without limitation, alfentanil, apomorphine, buprenorphine, butorphanol, codeine, dextropropoxyphene, diamorphine, dihydrocodeine, fentanyl, hydrocodone, hydromorphone, levorphanol, meperidine, meptazinol, methadone, morphine, nalbuphine, oxycodone, oxymorphone, pentazocine, propoxyphene, sufentanil, and tramadol.

In other embodiments, the active agent is a peptidyl drug substance include therapeutic peptides and proteins per se, whether naturally occurring, chemically synthesized, recombinantly produced, and/or produced by biochemical (e.g., enzymatic) fragmentation of larger molecules, and may contain the native sequence or an active fragment thereof. Specific peptidyl drugs include, for example and without limitation, the peptidyl hormones activin, amylin, angiotensin, atrial natriuretic peptide (ANP), calcitonin, calcitonin gene-related peptide, calcitonin N-terminal flanking peptide, ciliary neurotrophic factor (CNTF), corticotropin (adrenocorticotropin hormone, ACTH), corticotropin-releasing factor (CRF or CRH), epidermal growth factor (EGF), follicle-stimulating hormone (FSH), gastrin, gastrin inhibitory peptide (GIP), gastrin-releasing peptide, gonadotropin-releasing factor (GnRF or GNRH), growth hormone releasing factor (GRF, GRH), human chorionic gonadotropin (hCH), inhibin A, inhibin B, insulin, luteinizing hormone (LH), luteinizing hormone-releasing hormone (LHRH), α-melanocyte-stimulating hormone, J3-melanocyte-stimulating hormone, γ-melanocyte-stimulating hormone, melatonin, motilin, oxytocin (pitocin), pancreatic polypeptide, parathyroid hormone (PTH), placental lactogen, prolactin (PRL), prolactin-release inhibiting factor (PIF), prolactin-releasing factor (PRF), secretin, somatotropin (growth hormone, GH), somatostatin (SIF, growth hormone-release inhibiting factor, GIF), thyrotropin (thyroid-stimulating hormone, TSH), thyrotropin-releasing factor (TRH or TRF), thyroxine, vasoactive intestinal peptide (VIP), and vasopressin. Other peptidyl drug substances are the cytokines, e.g., colony stimulating factor 4, heparin binding neurotrophic factor (HBNF), interferon-.alpha., interferon .alpha.-2a, interferon α-2b, interferon α-n3, interferon-β, etc., interleukin-1, interleukin-2, interleukin-3, interleukin-4, interleukin-5, interleukin-6, etc., tumor necrosis factor, tumor necrosis factor-.alpha., granuloycte colony-stimulating factor (G-CSF), granulocyte-macrophage colony-stimulating factor (GM-CSF), macrophage colony-stimulating factor, midkine (MD), and thymopoietin. Still other peptidyl drug substances include endorphins (e.g., dermorphin, dynorphin, α-endorphin, β-endorphin, γ-endorphin, δ-endorphin, [Leu⁵]enkephalin, [Met⁵]enkephalin, substance P), kinins (e.g., bradykinin, potentiator B, bradykinin potentiator C, kallidin), LHRH analogues (e.g., buserelin, deslorelin, fertirelin, goserelin, histrelin, leuprolide, lutrelin, nafarelin, tryptorelin), and the coagulation factors, such as α₁-antitrypsin, α₂-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, and thrombomodulin and combinations thereof.

In still other embodiments, the active agent is a sex hormone including, for example and without limitation, progestins (progestogens), estrogens, and combinations thereof. Progestins include acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, fluorogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, progesterone, and trimgestone. Also included within this general class are estrogens, e.g.: estradiol (i.e., 1,3,5-estratriene-3,17β-diol, or “17β-estradiol”) and its esters, including estradiol benzoate, valerate, cypionate, heptanoate, decanoate, acetate and diacetate; 17.alpha.-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. In many contexts, e.g., in female contraception and in hormone replacement therapy (HRT), a combination of a progestin and estrogen is used, e.g., progesterone and 17β-estradiol. For HRT, an androgenic agent may be advantageously included as well. Androgenic agents for this purpose include, for example, dehydroepiandrosterone (DHEA; also termed “prasterone”), sodium dehydroepiandrosterone sulfate, 4-dihydrotestosterone (DHT; also termed “stanolone”), and testosterone, and pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters.

In yet other embodiments, the active agent is a stimulant, including active drug substances for treating narcolepsy, attention deficit disorder (ADD), and attention deficit hyperactivity disorder (ADHD) including, for example and without limitation, amphetamine, dexamphetamine, dexfenfluramine, mazindol, methylphenidate (including d-threo-methylphenidate or “dexmethylphenidate”), mondafinil, pemoline and sibutramine.

Other androgenic agents include, but are not limited to, androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, ethylestrenol, oxandrolone, nandrolone phenpropionate, nandrolone decanoate, nandrolone furylpropionate, nandrolone cyclohexane-propionate, nandrolone benzoate, nandrolone cyclohexanecarboxylate, stanozolol, dromostanolone, and dromostanolone propionate.

Suitable drugs for use as active agents are also listed in: Goodman and Gilman's The Pharmacological Basis of Therapeutics (11th Ed) (Laurence Brunton) (McGraw-Hill Professional) (2005); and 2008 Physicians' Desk Reference (Thomas Healthcare) (2007).

Suitable active agents include, but are not limited to: antineoplastic agents, examples of which are disclosed in U.S. Pat. Nos. 5,880,161, 5,877,206, 5,786,344, 5,760,041, 5,753,668, 5,698,529, 5,684,004, 5,665,715, 5,654,484, 5,624,924, 5,618,813, 5,610,292, 5,597,831, 5,530,026, 5,525,633, 5,525,606, 5,512,678, 5,508,277, 5,463,181, 5,409,893, 5,358,952, 5,318,965, 5,223,503, 5,214,068, 5,196,424, 5,109,024, 5,106,996, 5,101,072, 5,077,404, 5,071,848, 5,066,493, 5,019,390, 4,996,229, 4,996,206, 4,970,318, 4,968,800, 4,962,114, 4,927,828, 4,892,887, 4,889,859, 4,886,790, 4,882,334, 4,882,333, 4,871,746, 4,863,955, 4,849,563, 4,845,216, 4,833,145, 4,824,955, 4,785,085, 4,684,747, 4,618,685, 4,611,066, 4,550,187, 4,550,186, 4,544,501, 4,541,956, 4,532,327, 4,490,540, 4,399,283, 4,391,982, 4,383,994, 4,294,763, 4,283,394, 4,246,411, 4,214,089, 4,150,231, 4,147,798, 4,056,673, 4,029,661, 4,012,448;

psychopharmacological/psychotropic agents, examples of which are disclosed in U.S. Pat. Nos. 5,192,799, 5,036,070, 4,778,800, 4,753,951, 4,590,180, 4,690,930, 4,645,773, 4,427,694, 4,424,202, 4,440,781, 5,686,482, 5,478,828, 5,461,062, 5,387,593, 5,387,586, 5,256,664, 5,192,799, 5,120,733, 5,036,070, 4,977,167, 4,904,663, 4,788,188, 4,778,800, 4,753,951, 4,690,930, 4,645,773, 4,631,285, 4,617,314, 4,613,600, 4,590,180, 4,560,684, 4,548,938, 4,529,727, 4,459,306, 4,443,451, 4,440,781, 4,427,694, 4,424,202, 4,397,853, 4,358,451, 4,324,787, 4,314,081, 4,313,896, 4,294,828, 4,277,476, 4,267,328, 4,264,499, 4,231,930, 4,194,009, 4,188,388, 4,148,796, 4,128,717, 4,062,858, 4,031,226, 4,020,072, 4,018,895, 4,018,779, 4,013,672, 3,994,898, 3,968,125, 3,939,152, 3,928,356, 3,880,834, 3,668,210;

cardiovascular agents, examples of which are disclosed in U.S. Pat. Nos. 4,966,967, 5,661,129, 5,552,411, 5,332,737, 5,389,675, 5,198,449, 5,079,247, 4,966,967, 4,874,760, 4,954,526, 5,051,423, 4,888,335, 4,853,391, 4,906,634, 4,775,757, 4,727,072, 4,542,160, 4,522,949, 4,524,151, 4,525,479, 4,474,804, 4,520,026, 4,520,026, 5,869,478, 5,859,239, 5,837,702, 5,807,889, 5,731,322, 5,726,171, 5,723,457, 5,705,523, 5,696,111, 5,691,332, 5,679,672, 5,661,129, 5,654,294, 5,646,276, 5,637,586, 5,631,251, 5,612,370, 5,612,323, 5,574,037, 5,563,170, 5,552,411, 5,552,397, 5,547,966, 5,482,925, 5,457,118, 5,414,017, 5,414,013, 5,401,758, 5,393,771, 5,362,902, 5,332,737, 5,310,731, 5,260,444, 5,223,516, 5,217,958, 5,208,245, 5,202,330, 5,198,449, 5,189,036, 5,185,362, 5,140,031, 5,128,349, 5,116,861, 5,079,247, 5,070,099, 5,061,813, 5,055,466, 5,051,423, 5,036,065, 5,026,712, 5,011,931, 5,006,542, 4,981,843, 4,977,144, 4,971,984, 4,966,967, 4,959,383, 4,954,526, 4,952,692, 4,939,137, 4,906,634, 4,889,866, 4,888,335, 4,883,872, 4,883,811, 4,847,379, 4,835,157, 4,824,831, 4,780,538, 4,775,757, 4,774,239, 4,771,047, 4,769,371, 4,767,756, 4,762,837, 4,753,946, 4,752,616, 4,749,715, 4,738,978, 4,735,962, 4,734,426, 4,734,425, 4,734,424, 4,730,052, 4,727,072, 4,721,796, 4,707,550, 4,704,382, 4,703,120, 4,681,970, 4,681,882, 4,670,560, 4,670,453, 4,668,787, 4,663,337, 4,663,336, 4,661,506, 4,656,267, 4,656,185, 4,654,357, 4,654,356, 4,654,355, 4,654,335, 4,652,578, 4,652,576, 4,650,874, 4,650,797, 4,649,139, 4,647,585, 4,647,573, 4,647,565, 4,647,561, 4,645,836, 4,639,461, 4,638,012, 4,638,011, 4,632,931, 4,631,283, 4,628,095, 4,626,548, 4,614,825, 4,611,007, 4,611,006, 4,611,005, 4,609,671, 4,608,386, 4,607,049, 4,607,048, 4,595,692, 4,593,042, 4,593,029, 4,591,603, 4,588,743, 4,588,742, 4,588,741, 4,582,854, 4,575,512, 4,568,762, 4,560,698, 4,556,739, 4,556,675, 4,555,571, 4,555,570, 4,555,523, 4,550,120, 4,542,160, 4,542,157, 4,542,156, 4,542,155, 4,542,151, 4,537,981, 4,537,904, 4,536,514, 4,536,513, 4,533,673, 4,526,901, 4,526,900, 4,525,479, 4,524,151, 4,522,949, 4,521,539, 4,520,026, 4,517,188, 4,482,562, 4,474,804, 4,474,803, 4,472,411, 4,466,979, 4,463,015, 4,456,617, 4,456,616, 4,456,615, 4,418,076, 4,416,896, 4,252,815, 4,220,594, 4,190,587, 4,177,280, 4,164,586, 4,151,297, 4,145,443, 4,143,054, 4,123,550, 4,083,968, 4,076,834, 4,064,259, 4,064,258, 4,064,257, 4,058,620, 4,001,421, 3,993,639, 3,991,057, 3,982,010, 3,980,652, 3,968,117, 3,959,296, 3,951,950, 3,933,834, 3,925,369, 3,923,818, 3,898,210, 3,897,442, 3,897,441, 3,886,157, 3,883,540, 3,873,715, 3,867,383, 3,873,715, 3,867,383, 3,691,216, 3,624,126;

antimicrobial agents examples of which are disclosed in U.S. Pat. Nos. 5,902,594, 5,874,476, 5,874,436, 5,859,027, 5,856,320, 5,854,242, 5,811,091, 5,786,350, 5,783,177, 5,773,469, 5,762,919, 5,753,715, 5,741,526, 5,769,870, 5,707,990, 5,696,117, 5,684,042, 5,683,709, 5,656,591, 5,643,971, 5,643,950, 5,610,196, 5,608,056, 5,604,262, 5,595,742, 5,576,341, 5,554,373, 5,541,233, 5,534,546, 5,534,508, 5,514,715, 5,508,417, 5,464,832, 5,428,073, 5,428,016, 5,424,396, 5,399,553, 5,391,544, 5,385,902, 5,359,066, 5,356,803, 5,354,862, 5,346,913, 5,302,592, 5,288,693, 5,266,567, 5,254,685, 5,252,745, 5,209,930, 5,196,441, 5,190,961, 5,175,160, 5,157,051, 5,096,700, 5,093,342, 5,089,251, 5,073,570, 5,061,702, 5,037,809, 5,036,077, 5,010,109, 4,970,226, 4,916,156, 4,888,434, 4,870,093, 4,855,318, 4,784,991, 4,746,504, 4,686,221, 4,599,228, 4,552,882, 4,492,700, 4,489,098, 4,489,085, 4,487,776, 4,479,953, 4,477,448, 4,474,807, 4,470,994, 4,370,484, 4,337,199, 4,311,709, 4,308,283, 4,304,910, 4,260,634, 4,233,311, 4,215,131, 4,166,122, 4,141,981, 4,130,664, 4,089,977, 4,089,900, 4,069,341, 4,055,655, 4,049,665, 4,044,139, 4,002,775, 3,991,201, 3,966,968, 3,954,868, 3,936,393, 3,917,476, 3,915,889, 3,867,548, 3,865,748, 3,867,548, 3,865,748, 3,783,160, 3,764,676, 3,764,677;

anti-inflammatory agents examples of which are disclosed in U.S. Pat. Nos. 5,872,109, 5,837,735, 5,827,837, 5,821,250, 5,814,648, 5,780,026, 5,776,946, 5,760,002, 5,750,543, 5,741,798, 5,739,279, 5,733,939, 5,723,481, 5,716,967, 5,688,949, 5,686,488, 5,686,471, 5,686,434, 5,684,204, 5,684,041, 5,684,031, 5,684,002, 5,677,318, 5,674,891, 5,672,620, 5,665,752, 5,656,661, 5,635,516, 5,631,283, 5,622,948, 5,618,835, 5,607,959, 5,593,980, 5,593,960, 5,580,888, 5,552,424, 5,552,422, 5,516,764, 5,510,361, 5,508,026, 5,500,417, 5,498,405, 5,494,927, 5,476,876, 5,472,973, 5,470,885, 5,470,842, 5,464,856, 5,464,849, 5,462,952, 5,459,151, 5,451,686, 5,444,043, 5,436,265, 5,432,181, RE034918, 5,393,756, 5,380,738, 5,376,670, 5,360,811, 5,354,768, 5,348,957, 5,347,029, 5,340,815, 5,338,753, 5,324,648, 5,319,099, 5,318,971, 5,312,821, 5,302,597, 5,298,633, 5,298,522, 5,298,498, 5,290,800, 5,290,788, 5,284,949, 5,280,045, 5,270,319, 5,266,562, 5,256,680, 5,250,700, 5,250,552, 5,248,682, 5,244,917, 5,240,929, 5,234,939, 5,234,937, 5,232,939, 5,225,571, 5,225,418, 5,220,025, 5,212,189, 5,212,172, 5,208,250, 5,204,365, 5,202,350, 5,196,431, 5,191,084, 5,187,175, 5,185,326, 5,183,906, 5,177,079, 5,171,864, 5,169,963, 5,155,122, 5,143,929, 5,143,928, 5,143,927, 5,124,455, 5,124,347, 5,114,958, 5,112,846, 5,104,656, 5,098,613, 5,095,037, 5,095,019, 5,086,064, 5,081,261, 5,081,147, 5,081,126, 5,075,330, 5,066,668, 5,059,602, 5,043,457, 5,037,835, 5,037,811, 5,036,088, 5,013,850, 5,013,751, 5,013,736, 5,006,542, 4,992,448, 4,992,447, 4,988,733, 4,988,728, 4,981,865, 4,962,119, 4,959,378, 4,954,519, 4,945,099, 4,942,236, 4,931,457, 4,927,835, 4,912,248, 4,910,192, 4,904,786, 4,904,685 4,904,674, 4,904,671, 4,897,397, 4,895,953, 4,891,370, 4,870,210, 4,859,686, 4,857,644, 4,853,392, 4,851,412, 4,847,303, 4,847,290, 4,845,242, 4,835,166, 4,826,990, 4,803,216, 4,801,598, 4,791,129, 4,788,205, 4,778,818, 4,775,679, 4,772,703, 4,767,776, 4,764,525, 4,760,051, 4,748,153, 4,725,616, 4,721,712, 4,713,393, 4,708,966, 4,695,571, 4,686,235, 4,686,224, 4,680,298, 4,678,802, 4,652,564, 4,644,005, 4,632,923, 4,629,793, 4,614,741, 4,599,360, 4,596,828, 4,595,694, 4,595,686, 4,594,357, 4,585,755, 4,579,866, 4,578,390, 4,569,942, 4,567,201, 4,563,476, 4,559,348, 4,558,067, 4,556,672, 4,556,669, 4,539,326, 4,537,903, 4,536,503, 4,518,608, 4,514,415, 4,512,990, 4,501,755, 4,495,197, 4,493,839, 4,465,687, 4,440,779, 4,440,763, 4,435,420, 4,412,995, 4,400,534, 4,355,034, 4,335,141, 4,322,420, 4,275,064, 4,244,963, 4,235,908, 4,234,593, 4,226,887, 4,201,778, 4,181,720, 4,173,650, 4,173,634, 4,145,444, 4,128,664, 4,125,612, 4,124,726, 4,124,707, 4,117,135, 4,027,031, 4,024,284, 4,021,553, 4,021,550, 4,018,923, 4,012,527, 4,011,326, 3,998,970, 3,998,954, 3,993,763, 3,991,212, 3,984,405, 3,978,227, 3,978,219, 3,978,202, 3,975,543, 3,968,224, 3,959,368, 3,949,082, 3,949,081, 3,947,475, 3,936,450, 3,934,018, 3,930,005, 3,857,955, 3,856,962, 3,821,377, 3,821,401, 3,789,121, 3,789,123, 3,726,978, 3,694,471, 3,691,214, 3,678,169, 3,624,216;

immunosuppressive agents, examples of which are disclosed in U.S. Pat. Nos. 4,450,159, 4,450,159, 5,905,085, 5,883,119, 5,880,280, 5,877,184, 5,874,594, 5,843,452, 5,817,672, 5,817,661, 5,817,660, 5,801,193, 5,776,974, 5,763,478, 5,739,169, 5,723,466, 5,719,176, 5,696,156, 5,695,753, 5,693,648, 5,693,645, 5,691,346, 5,686,469, 5,686,424, 5,679,705, 5,679,640, 5,670,504, 5,665,774, 5,665,772, 5,648,376, 5,639,455, 5,633,277, 5,624,930, 5,622,970, 5,605,903, 5,604,229, 5,574,041, 5,565,560, 5,550,233, 5,545,734, 5,540,931, 5,532,248, 5,527,820, 5,516,797, 5,514,688, 5,512,687, 5,506,233, 5,506,228, 5,494,895, 5,484,788, 5,470,857, 5,464,615, 5,432,183, 5,431,896, 5,385,918, 5,349,061, 5,344,925, 5,330,993, 5,308,837, 5,290,783, 5,290,772, 5,284,877, 5,284,840, 5,273,979, 5,262,533, 5,260,300, 5,252,732, 5,250,678, 5,247,076, 5,244,896, 5,238,689, 5,219,884, 5,208,241, 5,208,228, 5,202,332, 5,192,773, 5,189,042, 5,169,851, 5,162,334, 5,151,413, 5,149,701, 5,147,877, 5,143,918, 5,138,051, 5,093,338, 5,091,389, 5,068,323, 5,068,247, 5,064,835, 5,061,728, 5,055,290, 4,981,792, 4,810,692, 4,410,696, 4,346,096, 4,342,769, 4,317,825, 4,256,766, 4,180,588, 4,000,275, 3,759,921;

immunomodulatory agents, examples of which are disclosed in U.S. Pat. Nos. 4,446,128, 4,524,147, 4,720,484, 4,722,899, 4,748,018, 4,877,619, 4,998,931, 5,049,387, 5,118,509, 5,152,980, 5,256,416, 5,468,729, 5,583,139, 5,604,234, 5,612,060, 5,612,350, 5,658,564, 5,672,605, 5,681,571, 5,708,002, 5,723,718, 5,736,143, 5,744,495, 5,753,687, 5,770,201, 5,869,057, 5,891,653, 5,939,455, 5,948,407, 6,006,752, 6,024,957, 6,030,624, 6,037,372, 6,037,373, 6,043,247, 6,060,049, 6,087,096, 6,096,315, 6,099,838, 6,103,235, 6,124,495, 6,153,203, 6,169,087, 6,255,278, 6,262,044, 6,290,950, 6,306,651, 6,322,796, 6,329,153, 6,344,476, 6,352,698, 6,365,163, 6,379,668, 6,391,303, 6,395,767, 6,403,555, 6,410,556, 6,412,492, 6,468,537, 6,489,330, 6,521,232, 6,525,035, 6,525,242, 6,558,663, 6,572,860;

analgesic agents, examples of which are disclosed in U.S. Pat. Nos. 5,292,736, 5,688,825, 5,554,789, 5,455,230, 5,292,736, 5,298,522, 5,216,165, 5,438,064, 5,204,365, 5,017,578, 4,906,655, 4,906,655, 4,994,450, 4,749,792, 4,980,365, 4,794,110, 4,670,541, 4,737,493, 4,622,326, 4,536,512, 4,719,231, 4,533,671, 4,552,866, 4,539,312, 4,569,942, 4,681,879, 4,511,724, 4,556,672, 4,721,712, 4,474,806, 4,595,686, 4,440,779, 4,434,175, 4,608,374, 4,395,402, 4,400,534, 4,374,139, 4,361,583, 4,252,816, 4,251,530, 5,874,459, 5,688,825, 5,554,789, 5,455,230, 5,438,064, 5,298,522, 5,216,165, 5,204,365, 5,030,639, 5,017,578, 5,008,264, 4,994,450, 4,980,365, 4,906,655, 4,847,290, 4,844,907, 4,794,110, 4,791,129, 4,774,256, 4,749,792, 4,737,493, 4,721,712, 4,719,231, 4,681,879, 4,670,541, 4,667,039, 4,658,037, 4,634,708, 4,623,648, 4,622,326, 4,608,374, 4,595,686, 4,594,188, 4,569,942, 4,556,672, 4,552,866, 4,539,312, 4,536,512, 4,533,671, 4,511,724, 4,440,779, 4,434,175, 4,400,534, 4,395,402, 4,391,827, 4,374,139, 4,361,583, 4,322,420, 4,306,097, 4,252,816, 4,251,530, 4,244,955, 4,232,018, 4,209,520, 4,164,514, 4,147,872, 4,133,819, 4,124,713, 4,117,012, 4,064,272, 4,022,836, 3,966,944;

cholinergic agents, examples of which are disclosed in U.S. Pat. Nos. 5,219,872, 5,219,873, 5,073,560, 5,073,560, 5,346,911, 5,424,301, 5,073,560, 5,219,872, 4,900,748, 4,786,648, 4,798,841, 4,782,071, 4,710,508, 5,482,938, 5,464,842, 5,378,723, 5,346,911, 5,318,978, 5,219,873, 5,219,872, 5,084,281, 5,073,560, 5,002,955, 4,988,710, 4,900,748, 4,798,841, 4,786,648, 4,782,071, 4,745,123, 4,710,508;

adrenergic agents, examples of which are disclosed in U.S. Pat. Nos. 5,091,528, 5,091,528, 4,835,157, 5,708,015, 5,594,027, 5,580,892, 5,576,332, 5,510,376, 5,482,961, 5,334,601, 5,202,347, 5,135,926, 5,116,867, 5,091,528, 5,017,618, 4,835,157, 4,829,086, 4,579,867, 4,568,679, 4,469,690, 4,395,559, 4,381,309, 4,363,808, 4,343,800, 4,329,289, 4,314,943, 4,311,708, 4,304,721, 4,296,117, 4,285,873, 4,281,189, 4,278,608, 4,247,710, 4,145,550, 4,145,425, 4,139,535, 4,082,843, 4,011,321, 4,001,421, 3,982,010, 3,940,407, 3,852,468, 3,832,470;

antihistamine agents, examples of which are disclosed in U.S. Pat. Nos. 5,874,479, 5,863,938, 5,856,364, 5,770,612, 5,702,688, 5,674,912, 5,663,208, 5,658,957, 5,652,274, 5,648,380, 5,646,190, 5,641,814, 5,633,285, 5,614,561, 5,602,183, 4,923,892, 4,782,058, 4,393,210, 4,180,583, 3,965,257, 3,946,022, 3,931,197;

steroidal agents, examples of which are disclosed in U.S. Pat. Nos. 5,863,538, 5,855,907, 5,855,866, 5,780,592, 5,776,427, 5,651,987, 5,346,887, 5,256,408, 5,252,319, 5,209,926, 4,996,335, 4,927,807, 4,910,192, 4,710,495, 4,049,805, 4,004,005, 3,670,079, 3,608,076, 5,892,028, 5,888,995, 5,883,087, 5,880,115, 5,869,475, 5,866,558, 5,861,390, 5,861,388, 5,854,235, 5,837,698, 5,834,452, 5,830,886, 5,792,758, 5,792,757, 5,763,361, 5,744,462, 5,741,787, 5,741,786, 5,733,899, 5,731,345, 5,723,638, 5,721,226, 5,712,264, 5,712,263, 5,710,144, 5,707,984, 5,705,494, 5,700,793, 5,698,720, 5,698,545, 5,696,106, 5,677,293, 5,674,861, 5,661,141, 5,656,621, 5,646,136, 5,637,691, 5,616,574, 5,614,514, 5,604,215, 5,604,213, 5,599,807, 5,585,482, 5,565,588, 5,563,259, 5,563,131, 5,561,124, 5,556,845, 5,547,949, 5,536,714, 5,527,806, 5,506,354, 5,506,221, 5,494,907, 5,491,136, 5,478,956, 5,426,179, 5,422,262, 5,391,776, 5,382,661, 5,380,841, 5,380,840, 5,380,839, 5,373,095, 5,371,078, 5,352,809, 5,344,827, 5,344,826, 5,338,837, 5,336,686, 5,292,906, 5,292,878, 5,281,587, 5,272,140, 5,244,886, 5,236,912, 5,232,915, 5,219,879, 5,218,109, 5,215,972, 5,212,166, 5,206,415, 5,194,602, 5,166,201, 5,166,055, 5,126,488, 5,116,829, 5,108,996, 5,099,037, 5,096,892, 5,093,502, 5,086,047, 5,084,450, 5,082,835, 5,081,114, 5,053,404, 5,041,433, 5,041,432, 5,034,548, 5,032,586, 5,026,882, 4,996,335, 4,975,537, 4,970,205, 4,954,446, 4,950,428, 4,946,834, 4,937,237, 4,921,846, 4,920,099, 4,910,226, 4,900,725, 4,892,867, 4,888,336, 4,885,280, 4,882,322, 4,882,319, 4,882,315, 4,874,855, 4,868,167, 4,865,767, 4,861,875, 4,861,765, 4,861,763, 4,847,014, 4,774,236, 4,753,932, 4,711,856, 4,710,495, 4,701,450, 4,701,449, 4,689,410, 4,680,290, 4,670,551, 4,664,850, 4,659,516, 4,647,410, 4,634,695, 4,634,693, 4,588,530, 4,567,000, 4,560,557, 4,558,041, 4,552,871, 4,552,868, 4,541,956, 4,519,946, 4,515,787, 4,512,986, 4,502,989, 4,495,102; the disclosures of all the above of which are herein incorporated by reference.

The drug moiety of the conjugate may be the whole drug or a binding fragment or portion thereof that retains its affinity and specificity for the active agent of interest while having a linkage site for covalent bonding to the vector protein ligand or linker. The conjugates of such drugs may be used for the same disorders, diseases, and indications as the drugs themselves.

Suitable cancer chemotherapeutic agents for use in the ladder frame polyether carrier molecule ligand based conjugates of the invention include all drugs which may be useful for treating brain tumors or other neoplasia in or around the brain, either in the free form, or, if not so useful for such tumors in the free form, then useful when linked to the ladder frame polyether carrier molecule ligand. Specific chemotherapeutic agents are cytotoxic chemotherapeutic agents, including but not limited to adriamycin (also known as doxorubicin), cisplatin, paclitaxel, analogs thereof, and other chemotherapeutic agents that demonstrate activity against tumors ex vivo and in vivo. Such chemotherapeutic agents also include alkylating agents, antimetabolites, natural products (such as vinca alkaloids, epidophyllotoxins, antibiotics, enzymes and biological response modifiers), topoisomerase inhibitors, microtubule inhibitors, spindle poisons, hormones and antagonists, and miscellaneous agents such as platinum coordination complexes, anthracendiones, substituted ureas, etc. Those of skill in the art will know of other chemotherapeutic agents.

Other suitable chemotherapeutic agents are those, which in the free form, demonstrate unacceptable systemic toxicity at desired doses. The general systemic toxicity associated with therapeutic levels of such agents is reduced by their linkage to a polycyclic polyether carrier molecule. Particularly are cardiotoxic agents that are useful therapeutics but are dose limited by cardiotoxicity. A classic example is adriamycin (also known as doxorubicin) and its analogs, such as daunorubicin.

Research Compounds

The invention also encompasses conjugates comprising at least one research compound linked directly to an escorter and methods for preparing these research compound conjugates. The present invention is also directed to detectably labeled probes which use these research compounds.

Other embodiments encompass research compounds that may be used as biological tools to label cellular internal organelles such as the endoplasmic reticulum, golgi body, mitochondria, and the like.

The research compound, which includes particular fluorescent label or detectable group, is not a critical aspect of the invention, providing it does not significantly interfere with the ability of the conjugate to cross biological membranes. The detectable group can be any material having a detectable physical or chemical property. Thus, a label is any composition detectable by, for example, spectroscopic, photochemical, biochemical, immunochemical, electrical, optical or chemical means.

Examples of research compounds suitable for use in the present invention include, but are not limited to, fluorophores, fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold, functionalized carbon chains, or colored glass or plastic beads (e.g., polystyrene, polypropylene, latex, etc.). Such research compounds can be used independent of any additives or formulated as set forth herein with appropriate, well known and readily available carriers, diluents and/or excipients to provide certain characteristics not available when such research compounds are used independently. The amount and type of such carriers, diluents and/or excipients are known by the ordinarily skilled artisan.

Suitable fluorophores include those which absorb and/or emit at wavelengths which are distinguishable from the excitation and emission maxima of the other solution components (such as proteins present in the sample) to minimize background fluorescence. Fluorophores which produce fluorescent light efficiently, i.e., those that are characterized by high absorptivity at the appropriate wavelength and high fluorescence quantum yields are acceptable for use hereunder.

In general, suitable are fluorophores which efficiently produce fluorescence upon excitation with light whose wavelength falls within a range of about 200 to about 1000 nanometers, specifically in the range of about 350-800 nanometers. Fluorophore moieties include fluorescent dyes having (a) a high extinction coefficient, at least about 10,000, specifically greater than 50,000; (b) sufficiently long excitation and emission wavelength maxima so that interference from natural fluorescence of the components in the sample to be assay will be minimized; and (c) high fluorescence intensity.

The fluorophore moieties may be cyclic, polycyclic, particularly polycyclic aromatic having at least two rings, and not more than about six rings, more usually not more than about five rings, where at least two of the rings are fused or connected with conjugated olefins. The aromatic compound may be carbocyclic or heterocyclic, particularly having from 1-3, more usually 1-2 nitrogen atoms as heteroannular atoms. Other heteroannular atoms may include oxygen and sulfur.

Further examples of suitable fluorophores include, but are not limited to, eosin, TRITC-amine, quinine, fluorescein W, acridine yellow, lissamine rhodamine, B sulfonyl chloride erythroscein, ruthenium (tris, bipyridinium), Texas Red, nicotinamide adenine dinucleotide, flavin adenine dinucleotide, etc. Chemiluminescent compounds suitable for use as labels include, but are not limited to, luciferin and 2,3-dihydrophthalazinediones, e.g., luminol. For a review of various labeling or signal producing systems that can be used in the methods of the present invention, see U.S. Pat. No. 4,391,904.

Enzymes suitable for use as labels include, but are not limited to, hydrolases, particularly phosphatases, esterases and glycosidases, or oxidotases, particularly peroxidases.

Examples of research compounds include BODIPY®, Cascade Blue®, 4-hydroxy benzhydride, 6,7-dimethoxy 4-coumarin, 7-methyl-4-coumarin, 2,3-diaminonaphthalene, biotin, 6-TAMRA, coumarin, biotin, rhodamine, fluorescein isothiocyanate, and fluorescein.

The research compound may be coupled directly or indirectly to a desired component of an assay according to methods well known in the art. As indicated above, a wide variety of research compounds can be used, with the choice of research compound dependant on sensitivity required, ease of conjugation with the desired component of the assay, stability requirements, available instrumentation, and disposal provisions. Non-radioactive, non-fluorescent markers are often visualized by indirect methods. Generally, a ligand molecule, for example biotin, is covalently bound to the escorter. The ligand then binds to another molecule, for example streptavidin, which is either inherently detectable or covalently bound to a signal system, such as a detectable enzyme, a fluorescent compound, or a chemiluminescent compound.

Linkers

The invention also encompasses conjugates comprising at least one research compound or biologically active compound linked to an escorter trough a linker.

For example, the conjugate may contain ester linkages that are stable at serum pH but hydrolyse to release the drug when exposed to intracellular pH. Other examples include amino acid linkers designed to be sensitive to cleavage by specific enzymes in the desired target organ. Exemplary linkers are set out in Blattler et al. Biochem. 24:1517-1524, 1985; King et al. Biochem. 25:5774-5779, 1986; Srinivasachar and Nevill, Biochem. 28:2501-2509, 1989, each of which is incorporated herein by reference in its entirety. Generally, the biologically active compound (“active agent”) will have a functional group that can be conveniently reacted with an aldehyde, alcohol or carboxylic acid, to generate, for example, an ester or amide. When a linker is used, the linker can contain an alkyl, aryl, polyethylene glycol, polypropylene glycol, hydrazide, and/or amino acid backbone, and further contain an amide, ether, ester, hydrazone, disulphide linkage or any combination thereof. Linkages containing amino acid, ether and amide bound components are generally stable under conditions of physiological pH, normally 7.4 in serum.

In other embodiments, the linker is from 1 to 30 atoms long with carbon chain atoms that may be substituted by heteroatoms independently selected from the group consisting of O, N, or S.

In some embodiments, the linker group is hydrophilic to enhance the solubility of the conjugate in body fluids. In some embodiments, the linker contains or is attached to the escorter or the protein agent by a functional group subject to attack by other lysosomal enzymes (e.g., enzymes not deficient in the target lysosome or a lysosomal enzyme not conjugated to the escorter). In some embodiments, the escorter and active agent are joined by a linker comprising amino acids or peptides, lipids, or sugar residues. In some embodiments, the escorter and active agent are joined by groups introduced synthetically or by post-translational modifications.

In other embodiments, active agent-linker intermediates are similar to what has been described previously, but comprise, for example, either an active ester that can react with free amine groups created on the escorter or a maleimide that can react with free thiols created on the escorter via a SATA reaction or through other groups to which the active agent may be attached.

In other embodiments, the linker group is a photolabile linker, microwave-labile linker or radio-labile linker. For example, the design of the conjugate is such that the escorter is covalently bound to the linker via a non-labile bond, and the active agent is then attached to the linker through a photolabile bond (X):

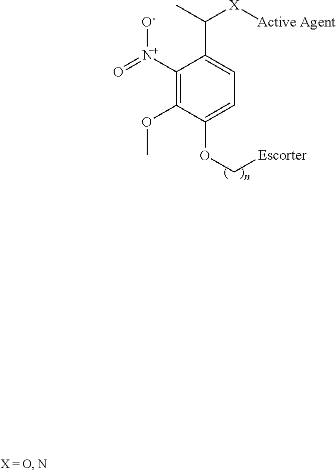

The irradiation with an appropriate wavelength of light releases the active agent by cleavage of the photolabile bond.

Representative functional group linkages, of which a linker may have one or more, are amides (—C(O)NR³—), ethers (—O—), thioethers (—S—), carbamates (—OC(O)NR³—), thiocarbamates (—OC(S)NR³—), ureas (—NR³C(O)NR³—), thioureas (—NR³C(S)NR³—), amino groups (—NR³—), carbonyl groups (—C(O)—), alkoxy groups (—O-alkylene-), etc. The linker may be homogenous or heterogeneous in its atom content (e.g., linkers containing only carbon atoms or linkers containing carbon atoms as well as one or more heteroatoms present on the linker. In another embodiment, the linker contains 1 to 25 carbon atoms and 0 to 15 heteroatoms selected from oxygen, NR³, sulfur, —S(O)— and —S(O)₂—, where R³ is hydrogen, alkyl or substituted alkyl. The linker may also be chiral or achiral, linear, branched or cyclic.

Intervening between the functional group linkages or bonds within the linker, the linker may further contain spacer groups including, but not limited to, spacers selected from alkyl, substituted alkyl, aryl, substituted aryl, cycloalkyl, substituted cycloalkyl, heteroaryl, substituted heteroaryl, heterocyclic, substituted heterocyclic, and combinations thereof. The spacer may be homogenous or heterogeneous in its atom content (e.g., spacers containing only carbon atoms or spacers containing carbon atoms as well as one or more heteroatoms present on the spacer. In another embodiment, the spacer contains 1 to 25 carbon atoms and 0 to 15 heteroatoms selected from oxygen, NR³, sulfur, —S(O)— and —S(O)₂—, where R³ is as defined above. The spacer may also be chiral or achiral, linear, branched or cyclic.

Non-limiting examples of spacers are straight or branched alkylene chains, phenylene, biphenylene, etc. rings, all of which are capable of carrying one or more than one functional group capable of forming a linkage with the active compound or research compound. One particular example of a polyfunctional linker-spacer group is lysine, which may link any of the active compounds to two polymer moieties via the two amino groups substituted on a O₄ alkylene chain. Other non-limiting examples include p-aminobenzoic acid and 3,5-diaminobenzoic acid which have 2 and 3 functional groups respectively available for linkage formation. Other such polyfunctional linkage plus spacer groups can be readily envisaged by one of skill in the art.

Reaction chemistries resulting in linker linkages are well known in the art. Such reaction chemistries involve the use of complementary functional groups on the linker, the escorter and the research compound. In another embodiment, the complementary functional groups on the linker are selected relative to the functional groups available on the escorter for bonding or which can be introduced onto the escorter for bonding. Again, such complementary functional groups are well known in the art. For example, reaction between a carboxylic acid of either the linker or the escorter and a primary or secondary amine of the escorter or the linker in the presence of suitable, well-known activating agents results in formation of an amide bond covalently linking the escorter moiety to the linker; reaction between an amine group of either the linker or the escorter group and a sulfonyl halide of the escorter or the linker results in formation of a sulfonamide bond covalently linking the escorter moiety to the linker; and reaction between an alcohol or phenol group of either the linker or the escorter and an alkyl or aryl halide of the escorter or the linker results in formation of an ether bond covalently linking the escorter group to the linker.

It is understood, of course, that if the appropriate substituents are found on the research compound then the optional linker may not be needed as there can be direct linkage of the escorter to the research compound.

Table 1 below illustrates numerous complementary reactive groups and the resulting bonds formed by reaction there between. One of ordinary skill in the art can select the appropriate solvents and reaction conditions to effect these linkages.

TABLE 1
Representative Complementary Binding Chemistries

| First Reactive Group | Second Reactive Group | Linkage |
|---|---|---|
| Hydroxyl | Isocyanate | Urethane |
| Amine | Epoxide | β-hydroxyamine |
| sulfonyl halide | Amine | Sulfonamide |
| Carboxyl | Amine | Amide |
| Hydroxyl | alkyl/aryl halide | Ether |
| Aldehyde (under reductive | Amine | Amine |
| amination conditions) | | |

Examples of linkers include, by way of example, the following —O—, —NR³—, —NR³C(O)O—, —OC(O)NR³—, —NR³C(O)—, —C(O)NR³—, —NR³C(O)NR³—, -alkylene-NR³C(O)O—, -alkylene-NR³C(O)NR³—, -alkylene-OC(O)NR³—, -alkylene-NR³—, -alkylene-O—, -alkylene-NR³C(O)—, -alkylene-C(O)NR³—, —NR³C(O)O-alkylene-, —NR³C(O)NR³-alkylene-, -00(O)NR³-alkylene, —NR³-alkylene-, —O-alkylene-, —NR³C(O)-alkylene-, —C(O)NR³-alkylene-, -alkylene-NR³C(O)O-alkylene-, -alkylene-NR³C(O)NR³-alkylene-, -alkylene-OC(O)NR³-alkylene-, -alkylene-NR³-alkylene-, alkylene-O-alkylene-, -alkylene-NR³C(O)-alkylene-, —C(O)NR³-alkylene-, —NR³C(O)O-alkyleneoxy-, —NR³C(O)NR³-alkyleneoxy-, —OC(O) NR³-alkyleneoxy, —NR³-alkyleneoxy-, —O-alkyleneoxy-, —NR³C(O)-alkyleneoxy-, —C(O)NR³-alkyleneoxy-, -alkyleneoxy-NR³C(O)O-alkyleneoxy- where R³ is as defined above and

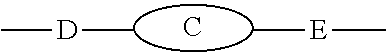

where

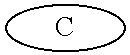

is selected from the group consisting of aryl, substituted aryl, cycloalkyl, substituted cycloalkyl, heteroaryl, substituted heteroaryl, heterocyclic and substituted heterocyclic, and D and E are independently selected from the group consisting of a bond, —O—, —CO—, —NR³—, —NR³C(O)O—, —OC(O)NR³—, —NR³C(O)—, —C(O)NR³—, —NR³C(O)NR³—, -alkylene-NR³C(O)O—, -alkylene-NR³C(O)NR³—, -alkylene-OC(O)NR³—, -alkylene-NR³—, -alkylene-O—, -alkylene-NR³C(O)—, alkylene-C(O)NR³—, —NR³C(O)O-alkylene-, —NR³C(O)NR³-alkylene-, —OC(O)NR³-alkylene-, —NR³-alkylene-, —O-alkylene-, —NR³C(O)-alkylene-, —NR³C(O)O-alkyleneoxy-, —NR³C(O)NR³-alkyleneoxy-, —OC(O)NR³-alkyleneoxy, —NR³-alkyleneoxy-, —O-alkyleneoxy-, —NR³C(O)-alkyleneoxy-, —C(O)NR³-alkyleneoxy-, -alkyleneoxy-NR³C(O)O-alkyleneoxy-, —C(O)NR³-alkylene-, -alkylene-NR³C(O)O-alkylene-, -alkylene-NR³C(O)NR³-alkylene-, -alkylene-OC(O)NR³-alkylene-, -alkylene-NR³-alkylene-, -alkylene-O-alkylene-, -alkylene-NR³C(O)-alkylene-, and —C(O)NR³-alkylene-, where R³ is as defined above.

Suitable alkylene groups in the above linkers include C₁-C₁₅ alkylene groups, such as C₁-C₆ alkylene groups and C₁-C₃ alkylene groups. Suitable heterocyclic groups include piperazinyl, piperidinyl, homopiperazinyl, homopiperidinyl, pyrrolidinyl, and imidazolidinyl. Suitable alkyleneoxy groups are —(CH₂—CH₂—O)₁₋₁₅—.

Compositions

The conjugates of this patent can be administered, for example, directly to biological membrane preparations, or to an animal in need of treatment via well known methods of administration including, for example, orally, topically, parenterally, by inhalation or spray or rectally in unit dosage formulations containing one or more pharmaceutically acceptable carriers, diluents or excipients. The term parenteral as used herein includes percutaneous, subcutaneous, intravascular (e.g., intravenous), intramuscular, or intrathecal injection or infusion techniques and the like.

The conjugates of the invention can be prepared in pharmaceutical preparations containing the conjugates themselves and one or more pharmaceutically acceptable adjuvant, carrier, diluent, excipient, solvent or other pharmaceutically acceptable substance(s) and/or vehicles (collectively, hereinafter “carriers”), or combinations thereof. Such carriers include those that facilitate administration of, prolong the shelf-life of, allow a particular mode of administration of, or provide or facilitate formulation of a particular dose of a conjugate of the present invention. The pharmaceutically acceptable carrier may be solid, liquid or aerosol. Examples of carriers, diluents and excipients that are suitable for such formulations include, for example and without limitation: fillers and extenders such as starch, sugars, mannitol, and silicic derivatives; binding agents such as carboxymethyl cellulose and other cellulose derivatives, alginates, gelatin and polyvinylpyrrolidone; moisturizing agents such as glycerol; disintegrating agents such as calcium carbonate and sodium bicarbonate; agents for retarding dissolution such as paraffin; resorption accelerators such as quarternary ammonium compounds; surface active agents such as cetyl alcohol, glycerol monostearate; absorptive carriers such as laolin and bentonite; and lubricants such as talc, calcium and magnesium stearate and solid polyethyl glycols.

Capsules and other protective mediums are suitable for the oral administration of the conjugates of the invention due to the protection afforded against hydrolysis in the gastrointestinal tract. When the present conjugates are to be administered peritoneally, they can be administered by subcutaneous, intramuscular or intravenous injections.

The present invention provides conjugates, compositions, and methods adapted for the site-specific/sustained delivery of a biologically active compound to its target.

The conjugates of the present invention can also be used in plant research and/or development via direct exposure of such conjugate(s) to one or more target species. These conjugates can also be formulated for use in applications to crops, insects, weeds, or other agricultural or target species using well known formulation techniques. For example and without limitation, the conjugates may be prepared as wettable powders, dry flowable formulations, liquids, suspensions, granules, emulsions, slow or controlled release formulations, and the like (see, e.g., U.S. Pat. Nos. 3,284,295; 4,389,238; 4,557,929; 6,307,850; and 7,163,687). The amount and type of adjuvants (including, for example, carries, diluents, excipients, solvents, surfactants and the like, and combinations thereof, used in the preparation of such formulations is product dependent but the processes for such preparation are well known in the art. Moreover, the amount of each conjugate equivalent to be applied for such uses will be dependent upon a variety of factors including, for example, environmental factors, stage of growth of the target species, density of the target species, location of the target species, recommendations or regulatory-labeled requirements, and the like. The method of application of the final formulation is typically dictated by a variety of factors including, for example, type of equipment available, the target species, the presence of non-target species relative to the target species, habitat, location of habitat relative to populated areas, and the like, and can be applied, for example, by ground, air, injection into irrigation systems, spreaders and the like.

The conjugates of the invention can also be prepared for use as biological tools. Conjugates suitable for use as such tools can be comprised of an escorter molecule coupled to an active agent and can be prepared by methods known in the art. The conjugates may also comprise an escorter covalently coupled indirectly to the active agent via a linker compound by bonds. Again, such conjugates can be prepared by methods known in the art. The linker component itself may be biologically active. The conjugates are designed to act as biological tools or in aiding visualization of biological processes, cellular organelles, etc. The methods of visualization include, but are not limited to, fluorescence, phosphorescence, colorimetric, etc processes.

Methods of Synthesis

In general, the escorter-active agent conjugates of the invention can be prepared using techniques known in the art. There are numerous approaches for the conjugation or chemical crosslinking of compounds and one skilled in the art can determine which method is appropriate for the active agent to be conjugated. The method employed must be capable of joining the active agent to the escorter, generally without altering the desired activity of the agent once delivered. Exemplary methods of conjugating the escorter to various active agents are set out in the Example section, below.

Methods for conjugating the escorter with the representative active agents set forth above may be readily accomplished by one of ordinary skill in the art.

The active agent and escorter can be coupled using a variety of reactions involving treating the active agent (or a protected derivative thereof) with the appropriate escorter molecule or an activated derivative thereof.

The escorter may contain lactone rings, alcohols, aldehydes, amine, amides, alkenes, and carboxylic acids. Modification and linkage of a lactone containing escorter to a desired functionality is described in Scheme 1 below. Those skilled in the art will recognize that through chemical manipulation of the lactone ring a variety of compounds, including, but not limited to, amines, amides and esters are easily accessible.

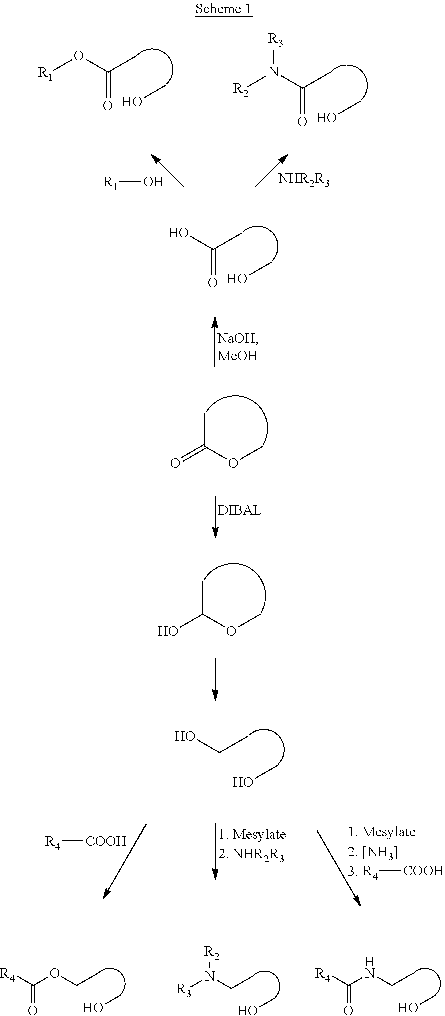

Modification and linkage of an aldehyde containing escorter is described in Scheme 2 below. Those skilled in the art will recognize that through chemical manipulation of the aldehyde, a variety of compounds, including, but not limited to, amines, amides, esters and hydrazides are easily accessible.

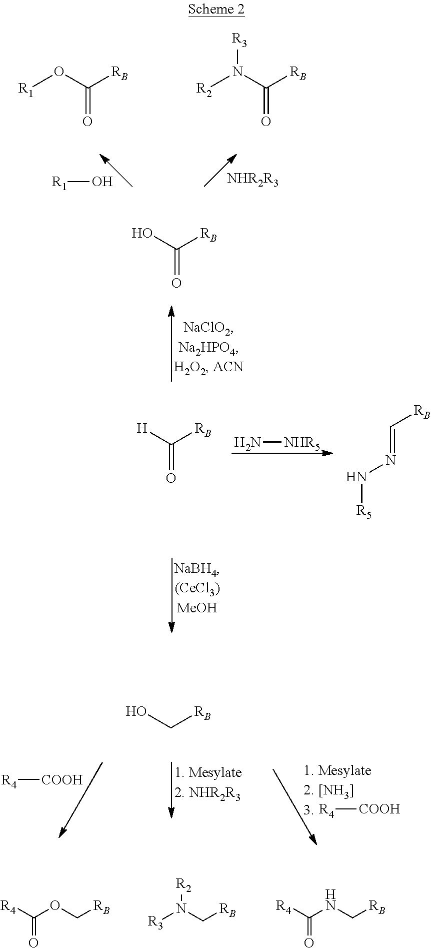

Methods of Using

This invention further pertains to methods for introducing one or more active agents into cells and across biological membranes. An effective amount, typically a pharmaceutically effective amount, is that amount necessary to prevent, treat, or reduce the symptoms associated with a particular condition or disease being treated. The specific dose of a conjugate administered according to this invention will, of course, be determined by the particular circumstances surrounding the case including, for example, the biologically active compound being used, the route of administration, the state of being of the patient, and the disease state being treated.

This invention also pertains to methods for manufacturing pharmaceutical preparations, including coupling an escorter to an active agent to form a prodrug, and then forming a pharmaceutical dose containing the prodrug and a pharmaceutically acceptable carrier.

Receptor binding assays of fluorescent brevetoxins, brevisins and brevenals demonstrated that the fluorescent-derivatives bound to rat brain synaptosomes with affinities similar to the parent compound (brevetoxin 2-10 nm, brevenal (400-800 nM or brevenal 200 to 400 nm). These results indicate that labeling of brevetoxins, brevisins and brevenals with large, cumbersome and sometimes charged molecules did not alter the binding of the brevetoxin and brevenal derivatives to their respective receptor binding sites. Fluorescent derivatives can replace antibody based visualization methods for brevetoxins and brevenals in cells and tissues.

In vitro cell based assays with mammalian cell lines (e.g. MCF7, SJCRH30, HEK and MDCK) showed that the fluorescently labeled polyethers did not accumulate on the cell surface, but rather fluorescently labeled polyethers were rapidly transported into the cells.

### EXAMPLES

The preparation of the compounds of the invention is illustrated further by the following examples, which are not to be construed as limiting the invention in scope or spirit to the specific procedures and compounds described in them. Representative methods for synthesizing compounds of the invention are presented below. It is understood that the nature of the substituents required for the desired active compound often determines the method of synthesis.

General Procedure A:

Escorter acid (1 eq) was dissolved in acetonitrile. To this solution, the following were sequentially added: triethylamine (3 eq), active agent amine or alcohol (1.1 eq), 2-bromo-1-ethylpyridinium tetrafluoroborate (BEP) (1.5 eq) and a catalytic amount of N,N-dimethylaminopyridine. The reaction mixture was stirred at room temperature overnight. Reaction progress was monitored using thin layer chromatography. The reaction mixture was partitioned between water and ethyl acetate. The organic layer was washed sequentially with water, a brine solution and then dried over sodium sulfate, filtered and evaporated under reduced pressure. The desired product was isolated using appropriate chromatographic conditions.

General Procedure B:

To a solution of escorter aldehyde (1 eq) in dimethylformamide was added active agent hydrazide (2 eq) and catalytic tungstophosphoric acid. The mixture was then heated at 60° C. for 4 hours. Reaction progress was monitored using thin layer chromatography. Solvents were removed in vacuo and the residue partitioned between dichloromethane and water. The organic fraction was evaporated under reduced pressure. The desired product was isolated using appropriate chromatographic conditions.

General Procedure C:

To a solution of N,N′-dicyclohexylcarbodiimide (DCC) (10 eq) in dichloromethane was added active agent acid (5 eq) and the mixture stirred for 15 minutes. To this was then added escorter alcohol (1 eq) and a catalytic amount of N,N-dimethylaminopyridine and the reaction stirred at room temperature for 3 hours. Reaction progress was monitored using thin layer chromatography. An equal volume of water was added to the reaction and the organic layer removed and concentrated under reduced pressure. The residue was then suspended in ethyl acetate and washed three times with water. The organic fraction was then concentrated under reduced pressure. The desired product was isolated using appropriate chromatographic conditions.

General Procedure D:

To a solution of N,N′-dicyclohexylcarbodiimide (DCC) (2 eq) in dichloromethane was added escorter acid (1 eq) and the mixture stirred for 15 minutes. To this was then added active agent amine or alcohol (2 eq) and a catalytic amount of N,N-dimethylaminopyridine and the reaction stirred at room temperature for 3 hours. Reaction progress was monitored using thin layer chromatography. An equal volume of water was added to the reaction and the organic layer removed and concentrated under reduced pressure. The residue was then suspended in ethyl acetate and washed three times with water. The organic fraction was then concentrated under reduced pressure. The desired product was isolated using appropriate chromatographic conditions.

General Procedure E:

To a solution of escorter aldehyde (1 eq) in anhydrous methanol was added active agent amine (2 eq) and catalytic tungstophosphoric acid. The mixture was then heated to reflux for 4 hours. Reaction progress was monitored using thin layer chromatography. Solvents were removed in vacuo and the residue partitioned between dichloromethane and water. The organic fraction was evaporated under reduced pressure. The desired product was isolated using appropriate chromatographic conditions.

### Examples 1-14

The following table lists examples of the invention. These examples can be represented by Formula I

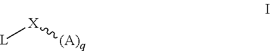

where R_{B} (“Escorter”) in the table represents L and “Active Agent” represents —X-(A)_{q}. Table 2 lists conjugates of the invention, i.e., combinations of R_{B} and Active Agent, and shows the specific Active Agents employed by the combinations. Structures for the R_{B} fragments are shown below in Table 3. Synthetic procedures for preparing the compounds of Table 2 are set forth after Table 3.

TABLE 2

| Example | Escorter, | | | Synthesis |
|---|---|---|---|---|
| No. | R_{B} | Active agent | [M + H]⁺ | procedure |
| 1 | R_{B1} | 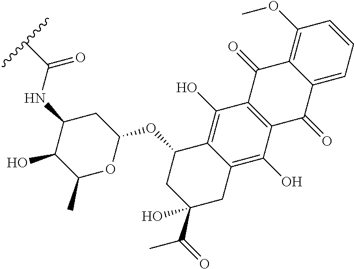 [SMILES: *C(=O)N[C@H]1C[C@H](O[C@H]2C[C@](O)(C(C)=O)Cc3c(O)c4c(c(O)c32)C(=O)c2c(OC)cccc2C4=O)O[C@@H](C)[C@H]1O] | 1421.6 | A |
| 2 | R_{B2} | 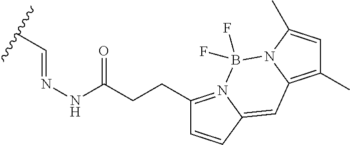 | 946.2 | B |
| 3 | R_{B3} | 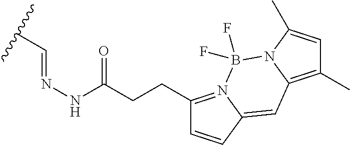 | 996.3 | B |
| 4 | R_{B1} | 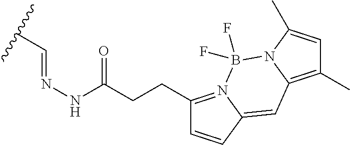 | 1184.4 | B |
| 5 | R_{B1} | 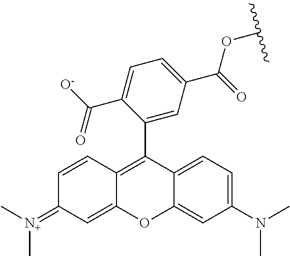 | 1310.2 | C |
| 6 | R_{B2} | 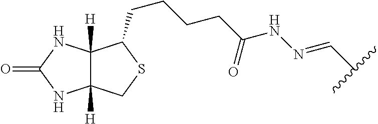 [SMILES: */C=N/NC(=O)CCCC[C@@H]1SC[C@@H]2NC(=O)N[C@@H]21] | 898.0 | B |
| 7 | R_{B3} | 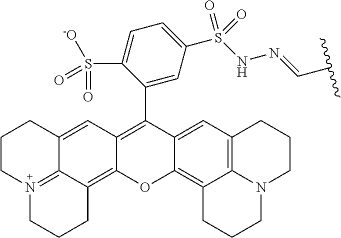 | 1310.2 | B |
| 8 | R_{B1} | 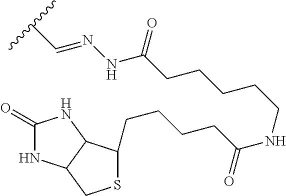 [SMILES: */C=N/NC(=O)CCCCCNC(=O)CCCCC1SCC2NC(=O)NC21] | 1249.4 | B |
| 9 | R_{B3} | 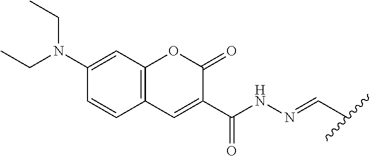 [SMILES: */C=N/NC(=O)c1cc2ccc(N(CC)CC)cc2oc1=O] | 965.4 | B |
| 11 | R_{B3} | 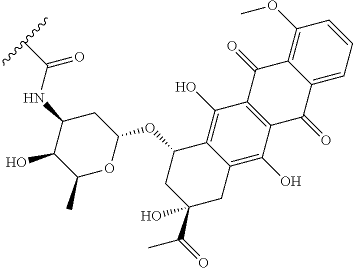 [SMILES: *C(=O)N[C@H]1C[C@H](O[C@H]2C[C@](O)(C(C)=O)Cc3c(O)c4c(c(O)c32)C(=O)c2c(OC)cccc2C4=O)O[C@@H](C)[C@H]1O] | 1233.0 | D |
| 10 | R_{B4} | 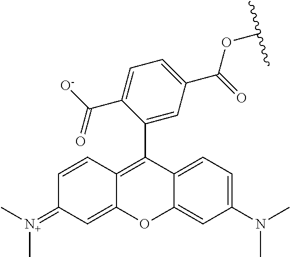 | 1314.3 | C |
| 12 | R_{B3} | 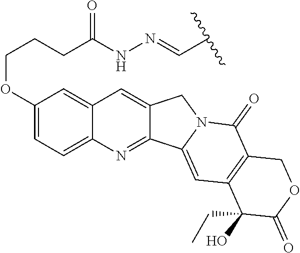 [SMILES: */C=N/NC(=O)CCCOc1ccc2nc3c(cc2c1)Cn1c-3cc2c(c1=O)COC(=O)[C@]2(O)CC] | 1154.1 | B |
| 13 | R_{B1} | 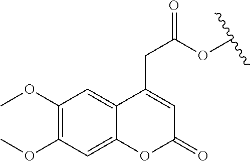 [SMILES: *OC(=O)Cc1cc(=O)oc2cc(OC)c(OC)cc12] | 1144.2 | C |
| 14 | R_{B1} | 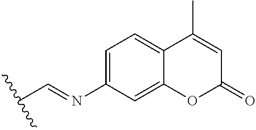 [SMILES: */C=N/c1ccc2c(C)cc(=O)oc2c1] | 1052.9 | E |

TABLE 3

| | |
|---|---|
| R_{B1} | 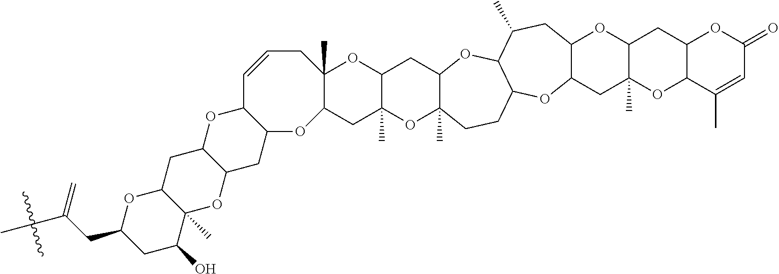 [SMILES: *C(=C)C[C@@H]1C[C@H](O)[C@]2(C)OC3CC4OC5C[C@]6(C)O[C@]7(C)CCC8OC9C[C@]%10(C)OC%11C(C)=CC(=O)OC%11CC%10OC9C[C@@H](C)C8OC7CC6O[C@@]5(C)C/C=C\C4OC3CC2O1] |
| R_{B2} | 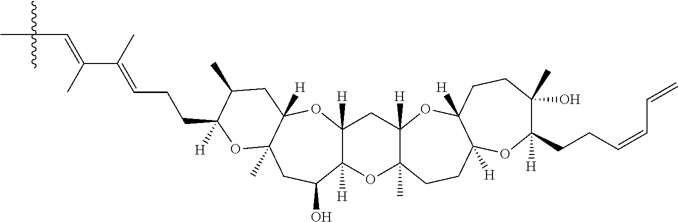 [SMILES: */C=C(C)/C(C)=C/CC[C@@H]1O[C@]2(C)C[C@H](O)[C@@H]3O[C@]4(C)CC[C@@H]5O[C@H](CC/C=C\C=C)[C@@](C)(O)CC[C@H]5O[C@H]4C[C@H]3O[C@H]2C[C@@H]1C] |
| R_{B3} | 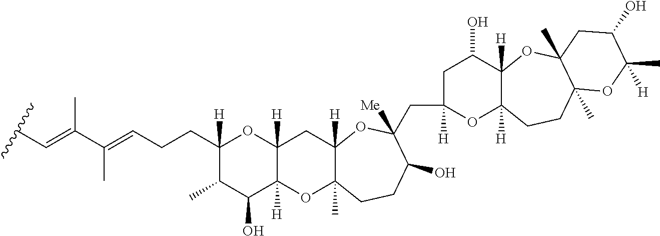 [SMILES: */C=C(C)/C(C)=C/CC[C@@H]1O[C@@H]2C[C@@H]3O[C@](C)(C[C@H]4C[C@H](O)[C@@H]5O[C@]6(C)C[C@H](O)[C@@H](C)O[C@@]6(C)CC[C@H]5O4)[C@@H](O)CC[C@@]3(C)O[C@H]2[C@@H](O)[C@@H]1C] |
| R_{B4} | 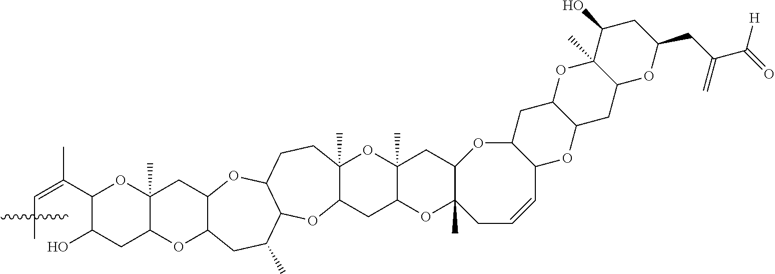 [SMILES: */C=C(/C)C1O[C@@]2(C)CC3OC4CC[C@@]5(C)O[C@@]6(C)CC7OC8CC9O[C@]%10(C)C(CC9OC8/C=C\C[C@]7(C)OC6CC5OC4[C@H](C)CC3OC2CC1O)O[C@H](CC(=C)C=O)C[C@@H]%10O] |
| R_{B5} | 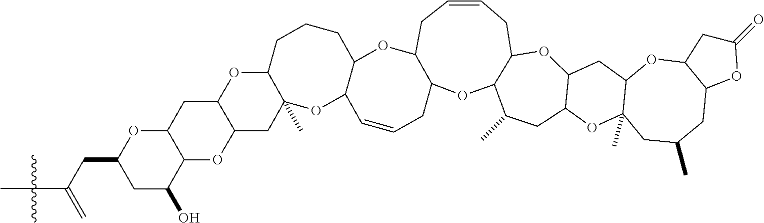 [SMILES: *C(=C)C[C@@H]1C[C@H](O)C2OC3C[C@]4(C)OC5/C=C\CC6OC7C(C/C=C\CC6OC5CCCC4OC3CC2O1)OC1CC2OC3CC(=O)OC3C[C@@H](C)C[C@@]2(C)OC1C[C@@H]7C] |
| R_{B6} | 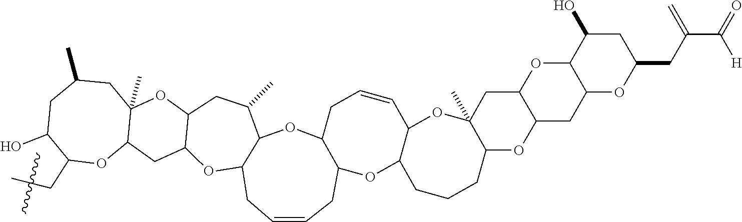 [SMILES: *CC1OC2CC3OC4C/C=C\CC5OC6CCCC7OC8CC9O[C@H](CC(=C)C=O)C[C@H](O)C9OC8C[C@]7(C)OC6/C=C\CC5OC4[C@@H](C)CC3O[C@]2(C)C[C@H](C)CC1O] |

### Example 1

Example 1 was prepared using general procedure A using BTX-B5 (R_{B1}) as the escorter acid and Daunorubicin as the active agent amine. The crude product was subjected to reverse phase HPLC using an 8 mm×250 mm 5 μm C₁₈ column. Eluent was 98:2 methanol:water at a flowrate of 3.4 mL/min and a detection wavelength of 215 nm. Example 1 was obtained as an orange solid in 41% yield. ¹H NMR (500 MHz, C₆D₆), δ ppm 0.91 (m, 8H), 1.01 (d, J=7 Hz, 3H), 1.06 (s, 3H), 1.14 (d, J=6 Hz, 4H), 1.33 (m, 19H), 1.48 (s, 3H), 1.69 (m, 7H), 1.83 (m, 2H), 1.98 (m, 6H), 2.13 (m, 4H), 2.19 (m, 1H), 2.31 (s, 2H), 2.40 (m, 0.5H), 2.47 (d, J=8 Hz, 1H), 2.72 (m, 1.5H), 2.81 (dd, J=13 Hz and 4 Hz, 0.5H), 2.98 (m, 2H), 3.07 (m, 1H), 3.15 (m, 1H), 3.23 (m, 1.5H), 3.33 (s, 2H), 3.39 (d, J=6 Hz, 1.5H), 3.42 (m, 1H), 3.53 (m, 2H), 3.63 (m, 1.5H), 3.74 (m, 0.5H), 4.00 (m, 0.5H), 4.08 (m, 1.5H), 4.17 (m, 0.5H), 4.41 (m, 0.5H), 5.10 (s, 1H), 5.14 (s, 0.5H), 5.50 (s, 1H), 5.55 (s, 1H), 5.59 (s, 1H), 5.80 (m, 1H), 5.92 (m, 0.5H), 5.99 (m, 0.5H), 6.34 (br s, 0.5H), 6.51 (d, J=9 Hz, 1H), 7.00 (s, 0.5H), 7.05 (t, J=8 Hz, 1H), 7.32 (s, 0.5H), 8.01 (d, J=8 Hz, 1H), 8.40 (m, 0.5H), 13.69 (s, 1H), 14.59 (s, 1H). (MS: M+H=1421.6, C₇₇H₉₇NO₂₄ requires 1420.59).

### Example 2

Example 2 was prepared using general procedure B using brevenal (R_{B2}) as the escorter aldehyde and 4,4-difluoro-5,7-dimethyl-4-bora-3a,4a-diaza-s-indacene-3-propionic acid, hydrazide (D2371 BODIPY®) as the active agent hydrazide. The crude product was subjected to reverse phase HPLC using a 4.6 mm×250 mm 5 μm O₁₈ column. Eluent was 90:10 acetonitrile:water with a flowrate of 1.4 ml/min and detection wavelength of 215 nM. Example 2 was isolated as an orange solid in 90% yield. ¹H NMR (500 MHz, CD₂Cl₂), δ ppm 0.91 (d, J=7 Hz, 3H), 1.05 (s, 3H), 1.12 (s, 3H), 1.13 (s, 3H), 1.20 (s, 1H), 1.25 (s, 2H), 1.33 (m, 1.5H), 1.40 (m, 1H), 1.47 (m, 1H), 1.52 (m, 4H), 1.56 (m, 1H), 1.59 (m, 1H), 1.69 (m, 3.5H), 1.75 (m, 2H), 1.80 (m, 4H), 1.86 (dd, J=15 Hz and 5 Hz, 1H), 1.96 (m, 3H), 2.03 (dt, J=12 Hz and 5 Hz, 1H), 2.13 (m, 2H), 2.19 (m, 2H), 2.24 (m, 3H), 2.30 (m, 2H), 2.52 (m, 3H), 2.65 (t, J=7 Hz, 0.5H), 3.03 (t, J=8 Hz, 1.5H), 3.13 (dd, J=12 Hz and 4 Hz, 1H), 3.17 (dd, J=11 Hz and 1 Hz, 1H), 3.26 (t, J=8 Hz, 2H), 3.29 (t, J=4 Hz, 2H), 3.36 (dd, J=10 Hz and 3 Hz, 1H), 3.49 (m, 1H), 3.59 (m, 1H), 3.69 (dd, J=12 Hz and 5 Hz, 1H), 3.98 (m, 1H), 5.08 (d, J=10 Hz, 1H), 5.18 (dd, J=17 Hz and 2 Hz, 1H), 5.46 (m, 1H), 5.82 (t, J=9 Hz, 1H), 6.02 (t, J=11 Hz, 1H), 6.13 (s, 0.7H), 6.15 (s, 0.3H), 6.22 (d, J=10 Hz, 1H), 6.31 (m, 1H), 6.64 (dt, J=11 Hz and 1 Hz, 0.5H), 6.67 (dd, J=11 Hz and 1 Hz, 0.5H), 6.91 (d, J=4 Hz, 1H), 7.12 (s, 0.7H), 7.14 (s, 0.3H). (MS: M+H=946.2, C₅₃H₇₅BF₂N₄O₈ requires 944.99).

### Example 3

Example 3 was prepared using general procedure B using brevisin (R_{B3}) as the escorter aldehyde and D2371 BODIPY® as the active agent hydrazide. Mixture cooled and diluted with water (8 mL). The crude mixture was then loaded onto a Strata-X C₁₈ cartridge. The cartridge was then washed sequentially with 4 column volumes each of water, 20% aqueous methanol, 80% aqueous methanol, methanol, and acetone. Desired product eluted in methanol fractions. HPLC purification was performed using an 8 mm×250 mm 5 pm C₃₀ column. Eluent was 55:45 acetonitrile:water with a flowrate of 3.4 ml/min and detection wavelength of 215 nM. Example 3 was isolated as an orange solid in 20% yield. ¹H NMR (500 MHz, CD₃OD), δ ppm 0.88 (m, 3H), 0.98 (m, 4.5H), 1.16 (m, 4.5H), 1.24 (m, 12H), 1.31 (m, 5.5H), 1.44 (m, 5.5H), 1.56 (m, 3H), 1.68 (m, 4H), 1.81 (m, 6H), 1.91 (m, 3.5H), 2.06 (m, 4.5H), 2.19 (m, 4H), 2.31 (m, 5H), 2.54 (m, 3H), 2.71 (m, 1H), 3.48 (m, 1H), 3.65 (m, 0.5H), 3.77 (m, 1H), 3.87 (m, 1H), 3.97 (m, 0.5H), 4.12 (m, 0.5H), 4.60 (m, 0.5H), 6.24 (m, 1H), 6.38 (m, 1H), 7.01 (m, 0.5H), 7.44 (m, 0.5H). (MS: M+H=996.3, C₆₃H₇₇BF₂N₄O₁₁ requires 995.01).

### Example 4

Example 4 was prepared using general procedure B, using PbTx-2 (R_{B1}) as the escorter aldehyde and D2371 BODIPY® as the active agent hydrazide. The crude product was subjected to reverse phase HPLC using a 4.6 mm×250 mm 5 μm C₁₈ column. Eluent was 90:10 methanol:water with a flowrate of 1.4 ml/min and detection wavelength of 215 nM. Example 4 was obtained as an orange solid in 98% yield. ¹H NMR (500 MHz, C₆D₆), δ ppm 0.82 (s, 3H), 1.06 (s, 3H), 1.14 (d, J=7 Hz, 3H), 1.28 (s, 3H), 1.48 (s, 3H), 1.50 (s, 3H), 1.54 (s, 3H), 1.64 (s, 3H), 1.79 (m, 1H), 1.83 (s, 3H), 1.89 (m, 4H), 1.99 (m, 3H), 2.12 (m, 6H), 2.29 (m, 5H), 2.67 (m, 5H), 2.77 (s, 1H), 2.85 (m, 1H), 2.95 (dd, J=12 Hz and 3 Hz, 1H), 3.00 (dd, J=14 Hz and 6 Hz, 1H), 3.12 (m, 1H), 3.22 (m, 2H), 3.36 (m, 4H), 3.55 (dd, J=13 Hz and 9 Hz, 1H), 3.65 (m, 1H), 3.72 (m, 2H), 3.85 (m, 4H), 3.95 (m, 1H), 4.21 (dd, J=12 Hz and 3 Hz, 1H), 4.27 (dd, J=12 Hz and 4 Hz, 1H), 4.42 (m, 1H), 5.23 (s, 1H), 5.57 (s, 1H), 5.68 (s, 1H), 5.70 (s, 1H), 5.93 (m, 1H), 6.12 (q, J=5 Hz, 1H), 6.54 (s, 1H), 6.57 (d, J=4 Hz, 1H), 6.70 (d, J=4 Hz, 1H), 6.95 (s, 1H), 9.40 (s, 1H). (MS: M+H=1184.4, C₆₄H₈₅BF₂N₄O₁₄ requires 1183.19).

### Example 5

Example 5 was prepared using general procedure C, using 6-carboxytetramethylrhodamine (6-TAMRA) as the active agent acid and PbTx-3 (R_{B1}) as the escortin alcohol. Example 5 was purified using a flash column packed with LH-20. The mobile phase was 100% methanol. Example 5 was isolated as a pink compound in approximately 80% yield. ¹H NMR (500 MHz, CD₂Cl₂), δ ppm 0.88 (m, 1H), 0.92 (t, J=7 Hz, 2H), 1.06 (d, J=7 Hz, 3H), 1.20 (s, 3H), 1.24 (m, 3H), 1.32 (m, 18H), 1.44 (m, 2H), 1.56 (m, 2H), 1.66 (m, 6H), 1.79 (m, 5H), 1.89 (m, 1H), 1.98 (s, 3H), 2.05 (m, 1H), 2.12 (m, 1.5H), 2.19 (m, 2H), 2.26 (dt, J=11 Hz and 4 Hz, 0.5H), 2.34 (m, 1H), 2.46 (m, 1H), 2.89 (s, 0.2H), 2.91 (s, 0.8H), 2.98 (m, 1H), 3.05 (t, J=7 Hz, 0.5H), 3.09 (m, 0.5H), 3.12 (m, 0.5H), 3.15 (m, 1H), 3.17 (m, 0.5H), 3.29 (m, 9H), 3.33 (m, 2H), 3.35 (m, 3H), 3.39 (m, 2H), 3.43 (m, 0.5H), 3.59 (m, 0.5H), 3.65 (m, 1H), 3.69 (s, 0.5H), 3.79 (m, 0.5H), 3.88 (dd, J=9 Hz and 3 Hz, 0.5H), 3.94 (dd, J=12 Hz and 4 Hz, 0.5H), 4.00 (m, 1.5H), 4.07 (m, 0.5H), 4.31 (d, J=11 Hz, 0.5H), 4.86 (q, J=13 Hz, 1H), 5.10 (m, 0.5H), 5.22 (s, 0.5H), 5.72 (t, J=2 Hz, 0.5H), 5.77 (t, J=5 Hz, 1H), 6.81 (m, 1.5H), 6.87 (m, 1.5H), 7.13 (m, 1H), 8.38 (dd, J=8 Hz and 2 Hz, 0.5H), 8.44 (m, 0.5H). (MS: M+H=1310.2, C₇₅H₉₂N₂O₁₈ requires 1309.54).

### Example 6

Example 6 was prepared using general procedure B, using brevenal (R_{B2}) as the escorter aldehyde and 5-((3aS,4S,6aR)-2-oxohexahydro-1H-thieno[3,4-d]imidazol-4-yl)pentanehydrazide (biotin hydrazide) as the active agent hydrazide. HPLC purification was performed using a 4.6 mm×250 mm 5 μm C₁₈ column. Eluent was 92% methanol in water with a flowrate of 1.4 ml/min and detection wavelength of 215 nM. The desired product was obtained as a yellow solid in 28% yield). ¹H NMR (500 MHz, CD₃OD) δ ppm 0.88 (m, 2H), 0.95 (m, 2H), 1.02 (s, 3H), 1.11 (s, 3H), 1.17 (br s, 3H), 1.27 (br s, 4H), 1.37 (m, 2H), 1.48 (m, 3H), 1.63 (m, 4H), 1.75 (br s, 7H), 1.84 (br s, 4H), 2.03 (m, 5H), 2.14 (m, 1H), 2.24 (m, 3H), 2.36 (m, 1H), 2.68 (m, 1H), 2.91 (m, 1H), 3.21 (m, 3H), 3.53 (m, 1H), 3.71 (m, 1H), 3.96 (m, 1H), 4.07 (m, 1H), 4.30 (m, 1H), 4.48 (m, 1H), 5.08 (m, 1H), 5.18 (d, J=18.31 Hz, 1H), 5.43 (m, 1H), 5.89 (m, 1H), 6.03 (m, 1H), 6.70 (m, 1H), 8.21 (br s, 1H). (MS: M+H=898.0, C₄₉H₇₆N₄O₉S requires 897.21).

### Example 7

Example 7 was prepared using general procedure B, using brevisin (R_{B3}) as the escorter aldehyde and sulforhodamine 101(Texas Red) as the active agent hydrazide. Crude mixture was then loaded onto a Strata-X C18 cartridge. The cartridge was then washed sequentially with 20 mL each of water, 20%, 40%, 60%, 80% methanol in water, methanol and acetone. Example 7 eluted in the 60% fraction. Solvents evaporated to give example 7 as a purple solid in 44% yield. ¹H NMR (500 MHz, CD₃OD) δ ppm 0.91 (d, J=6.41 Hz, 9H), 1.12 (m, 9H), 1.21 (m, 6H), 1.32 (m, 20H), 1.60 (m, 6H), 1.77 (m, 3H), 1.89 (s, 5H), 1.94 (m, 3H), 2.07 (m, 3H), 2.17 (m, 4H), 2.66 (s, 4H), 2.90 (m, 4H), 3.07 (t, J=5.50 Hz, 3H), 3.49 (t, J=5.50 Hz, 3H), 3.54 (t, J=5.50 Hz, 3H), 3.60 (s, 1H), 3.66 (m, 2H), 6.68 (s, 1H), 7.28 (d, J=7.63 Hz, 1H), 8.05 (dd, J=8.24 Hz and 1.83 Hz, 1H), 8.17 (m, 1H), 8.55 (s, 1H), 8.70 (d, J=1.83 Hz, 1H). MS: M+H=1310.2, C₇₀H₉₂N₄O₁₆S₂ requires 1309.63).

### Example 8

Example 8 was prepared using general procedure B, using PbTx-2 (R_{B1}) as the escorter aldehyde and N-(6-hydrazinyl-6-oxohexyl)-5-(2-oxohexahydro-1H-thieno[3,4-d]imidazol-4-yl)pentanamide (Long Arm Biotin) as the active agent hydrazide. HPLC purification was performed using a 4.6 mm×250 mm 5 μm C₁₈ column. Eluent was 88:12 methanol:water with a flowrate of 3.4 ml/min and detection wavelength of 215 nM. Example 8 was isolated as a white solid in 32% yield. ¹H NMR (500 MHz, C₆D₆) δ ppm 0.90 (m, 3H), 1.00 (d, J=7.02 Hz, 3H), 1.11 (m, 2H), 1.14 (s, 2H), 1.34 (s, 3H), 1.37 (s, 3H), 1.40 (s, 3H), 1.48 (s, 2H), 1.53 (s, 1H), 1.55 (s, 1H), 1.75 (m, 5H), 1.85 (m, 3 H), 1.96 (m, 3H), 2.05 (m, 2H), 2.12 (m, 2H), 2.20 (m, 2H), 2.52 (m, 1H), 2.60 (m, 2 H), 2.66 (m, 2H), 2.80 (m, 1H), 2.97 (m, 1H), 3.09 (m, 1H), 3.22 (m, 3H), 3.47 (m, 2 H), 3.53 (m, 1H), 3.58 (m, 1H), 3.66 (m, 2H), 3.73 (m, 1H), 3.78 (m, 1H), 4.09 (m, 1H), 4.15 (m, 1H), 4.31 (m, 1H), 5.24 (m, 1H), 5.53 (m, 1H), 5.58 (m, 1H), 5.96 (m, 1 H), 7.56 (m, 1H), 7.87 (m, 1H). (MS: M+H=1249.4, C₆₆H₉₇N₅O₁₆S requires 1248.57).

### Example 9

Example 9 was prepared using general procedure B, using brevisin (R_{B3}) as the escorter aldehyde and 7-(diethylamino)-2-oxo-2H-chromene-3-carbohydrazide (D355) as the active agent hydrazide. Product purified on HPLC using a 10 mm×250 mm 5 μm phenylhexy column. Eluent was 90:10 methanol:water with a flowrate of 3.4 mL/min and detection wavelength of 215 nm. Example 9 was isolated as a yellow solid in 51% yield. ¹H NMR (500 MHz, CD₃OD) δ ppm 0.74 (d, J=7.02 Hz, 3H), 0.92 (d, J=6.10 Hz, 3H), 0.99 (br s, 3H), 1.00 (s, 6H), 1.01 (s, 3H), 1.21 (m, 4H), 1.33 (m, 2H), 1.44 (m, 3H), 1.54 (m, 3H), 1.60 (d, J=5.19 Hz, 2H), 1.68 (m, 3H), 1.83 (br s, 1H), 1.86 (s, 2H), 2.06 (q, J=7.00 Hz, 2H), 2.90 (ddd, J=21.06 Hz, 9.77 Hz and 4.88 Hz, 1H), 2.96 (dd, J=8.85 Hz and 1.83 Hz, 1H), 3.09 (br s, 1H), 3.13 (m, 2H), 3.23 (dd, J=12.21 hz and 3.97 Hz, 1H), 3.40 (m, 1H), 3.53 (m, 2H), 3.64 (m, 2H), 3.72 (br s, 1H), 3.86 (d, J=2.44 Hz, 1H), 5.72 (t, J=7.60 Hz, 1H), 6.19 (d, J=10.38 Hz, 1H), 6.34 (d, J=1.83 Hz, 1H), 6.59 (d, J=8.24 Hz, 1H), 6.60 (m, 1H) 7.33 (d, J=8.85 Hz, 1H), 8.14 (d, J=9.77 Hz, 1H), 8.44 (s, 1H). (MS: M+H=965.4, C₅₃H₇₇N₃O₁₃ requires 964.19).

### Example 10

Example 10 was prepared using general procedure C, using 6-TAMRA as the active agent acid and Open A-ring PbTx-2 (R_{B4}) as the escortin alcohol. Example 10 was purified using a micro silica column. The column was prepared by packing a glass pipette with ˜1 inch of silica. The crude product was loaded with DCM and the column was eluted with 10 mL of 5% methanol in DCM, followed by 10 mL of 10% methanol in DCM. Example 10 eluted in the 5% fractions. The solvents were evaporated to give example 10 as a purple solid in 42% yield. ¹H NMR (500 MHz, CD₂Cl₂) δ ppm 0.91 (d, J=7.02 Hz, 3H), 1.01 (d, J=6.41 Hz, 4H), 1.15 (s, 4H), 1.17 (s, 2H), 1.20 (s, 5H), 1.26 (s, 12H), 1.36 (m, 8H), 1.48 (m, 5H), 1.62 (m, 12H), 1.75 (m, 10H), 1.90 (br s, 8 H), 2.02 (m, 4H), 2.15 (m, 3H), 2.31 (m, 2H), 2.42 (m, 2H), 2.65 (s, 1H), 3.02 (m, 4H), 3.20 (m, 2H), 3.37 (br. s., 3H), 3.60 (m, 4H), 3.77 (br s, 2H), 3.89 (m, 3H), 3.95 (m, 6H), 4.28 (m, 2H), 4.42 (m, 3H), 5.74 (d, J=4.27 Hz, 2H), 6.08 (s, 1H), 6.33 (s, 1H), 6.79 (d, J=2.14 Hz, 3H), 6.88 (m, 3H), 7.11 (d, J=0.61 Hz, 2H), 7.41 (d, J=9.77 Hz, 1H), 7.49 (s, 1H), 7.80 (dd, J=28.38 Hz and 8.85 Hz, 1H), 7.92 (s, 1H), 8.33 (d, J=10.38 Hz, 1H), 8.42 (d, J=7.63 Hz, 1H), 9.50 (s, 1H). (MS: M+H=1314.3, C₇₅H₉₆N₂O₁₈ requires 1313.57).

### Example 11

Example 11 was prepared using general procedure D, using brevisin acid (R_{B3}) as the escortin acid and duanorubicin as the active agent amine. Example 11 was purified using HPLC with an 8×250 mm 5 μm phenylhexyl column. Eluent was 98:2 methanol:water plus 0.1% formic acid at a flowrate of 3.4 ml/min. Detection wavelength was 215 nm. Example 11 was isolated as a green solid in 7% yield. ¹H NMR (500 MHz, CD₂Cl₂) δ ppm 0.93 (d, J=7.32 Hz, 3H), 1.14 (d, J=5.80 Hz, 3H), 1.19 (s, 3H), 1.20 (s, 3H), 1.21 (s, 3H), 1.26 (s, 10H), 1.59 (m, 12H), 1.76 (s, 5H), 1.84 (m, 5H), 2.09 (m, 3H), 2.18 (s, 3H), 2.23 (m, 2H), 2.33 (m, 1H), 2.37 (m, 1H), 2.41 (s, 3H), 2.55 (s, 1H), 2.99 (d, J=18.31 Hz, 1H), 3.20 (m, 3H), 3.33 (m, 2H), 3.42 (m, 2H), 3.63 (m, 3H), 3.73 (t, J=2.70 Hz, 1H), 3.76 (m, 1H), 3.82 (m, 1H), 3.94 (d, J=7.02 Hz, 1H), 4.03 (s, 3H), 4.07 (m, 1H), 4.17 (m, 1H), 4.26 (q, J=7.30 Hz, 1H), 5.30 (m, 1H), 5.49 (m, 1H), 5.73 (m, 1H), 5.83 (t, J=7.30 Hz, 1H), 5.90 (d, J=8.24 Hz, 1H), 7.41 (d, J=8.54 Hz, 1H), 7.79 (t, J=8.50 Hz, 1H), 8.02 (d, J=7.32 Hz, 1H), 13.31 (s, 1H), 14.05 (s, 1H). (MS: M+H=1233.0, C₆₆H₈₉NO₂₁ requires 1232.41).

### Example 12

Example 12 was prepared using general procedure B, using brevisin (R_{B3}) as the escorter aldehyde and 4-(10-camptothecin)oxybutane-hydrazide as the active agent hydrazide. Product purified on HPLC using a 10 mm×250 mm 5 μm phenylhexyl column using 88:12 methanol:water at a flowrate of 3.4 mL/min as eluent and monitoring at 254 nm. Example 12 was isolated as an off white solid in 34% yield. ¹H NMR (500 MHz, CD₃OD) δ ppm 0.94 (m, 3H), 1.01 (m, 3H), 1.14 (d, J=5.80 Hz, 3H), 1.21 (m, 8H), 1.27 (br s, 2H), 1.39 (m, 2H), 1.47 (m, 2H), 1.54 (m, 2H), 1.65 (m, 2H), 1.71 (m, 1H), 1.77 (m, 4H), 1.86 (s, 2H), 1.91 (s, 2H), 1.94 (m, 2H), 1.98 (s, 2H), 2.07 (m, 1H), 2.25 (d, J=7.32 Hz, 4H), 2.52 (t, J=7.90 Hz, 1H), 2.88 (t, J=7.00 Hz, 1H), 3.11 (m, 1H), 3.17 (dt, J=9.46H and 2.70 Hz, 2H), 3.35 (s, 3H), 3.44 (dd, J=12.51 Hz and 4.88 Hz, 2H), 3.62 (m, 2H), 3.75 (q, J=3.10 Hz, 2H), 3.86 (m, 2H), 3.94 (m, 1H), 4.08 (m, 2H), 4.22 (m, 3H), 5.18 (s, 3H), 5.33 (m, 2H), 5.43 (m, 3H), 5.57 (m, 1H), 5.87 (t, J=7.60 Hz, 1H), 6.34 (d, J=9.77 Hz, 1H), 7.27 (s, 1H), 7.43 (m, 2H), 7.57 (d, J=7.63 Hz, 2H), 7.98 (dd, J=8.54 Hz and 2.44 Hz, 1H), 8.16 (m, 1 H), 8.36 (s, 1H). (MS: M+H=1154.1, C₆₃H₈₄N₄O₁₆ requires 1153.36).

### Example 13

Example 13 was prepared using general procedure C, using 6,7-dimethoxy-4-acetic acid coumarin as the active agent acid and PbTx-3 (R_{B1}) as the escortin alcohol. The crude product was subjected to reverse phase HPLC using a 10 mm×250 mm 5 μm C₁₈ column. Eluent was a gradient of acetonitrile:water (85-95%) with a flowrate of 3.4 ml/min and detection wavelength of 215 nM. Example 13 was isolated as an off-white solid in 61% yield. ¹H NMR (500 MHz, CD₂Cl₂), δ ppm 1.02 (d, J=7.05 Hz, 4H), 1.26 (s, 3H), 1.27 (s, 3H), 1.28 (s, 3H), 1.43 (m, 2H), 1.52 (m, 4H), 1.59 (m, 1H), 1.65 (m, 4H), 1.75 (m, 4H), 1.86 (q, J=11.90 Hz, 1H), 1.94 (s, 3H), 2.03 (m, 3H), 2.12 (m, 2H), 2.22 (m, 2H), 2.30 (m, 1H), 2.44 (m, 1H), 2.60 (s, 1H), 2.91 (m, 2H), 3.11 (ddd, J=27.72 Hz, 12.49 Hz and 3.89 Hz, 3H), 3.29 (d, J=3.49 Hz, 3H), 3.36 (s, 2H), 3.56 (q, J=8.00 Hz, 1H), 3.74 (br s, 1H), 3.77 (s, 2H), 3.82 (d, J=4.41 Hz, 1H), 3.85 (m, 5H), 3.91 (m, 4H), 3.96 (dquin, J=11.10 Hz and 4.20 Hz, 2H), 4.27 (d, J=10.77 Hz, 1H), 4.60 (q, J=9.00 Hz, 2H), 4.98 (s, 1H), 5.04 (d, J=1.15 Hz, 1H), 5.68 (t, J=2.10 Hz, 1H), 5.74 (m, 2H), 6.22 (s, 1H), 6.86 (s, 1H), 6.97 (s, 1H). (MS: M+H=1144.2, C₆₃H₈₂O₁₉ requires 1143.31).

### Example 14

Example 14 was prepared using general procedure E using PbTx-2 (R_{B1}) as the escorter aldehyde and 7-amino-4-methyl coumarin as the active agent amine. The crude product was subjected to reverse phase HPLC using a 10 mm×250 mm 5 μm C₁₈ column. Eluent was a gradient of acetonitrile:water (97-100%) with a flowrate of 3.4 ml/min and detection wavelength of 215 nM. Example 14 was isolated as a white solid in 10% yield. ¹H NMR (500 MHz, CD₂Cl₂) δ ppm 1.01 (d, J=6.87 Hz, 5H), 1.15 (s, 5H), 1.25 (m, 20H), 1.46 (m, 4H), 1.61 (m, 8H), 1.74 (m, 6H), 1.86 (m, 3H), 1.93 (m, 4H), 2.02 (m, 4H), 2.11 (m, 3H), 2.20 (m, 3H), 2.31 (m, 2H), 2.46 (q, J=8.20 Hz, 1H), 2.78 (d, J=1.83 Hz, 1H), 3.09 (m, 6H), 3.29 (m, 7H), 3.41 (m, 1H), 3.55 (m, 2H), 3.62 (q, J=8.50 Hz, 1H), 3.85 (m, 1H), 3.90 (dd, J=8.94 Hz and 4.35 Hz, 1H), 3.98 (m, 3H), 4.18 (m, 2H), 4.26 (d, J=10.77 Hz, 1H), 5.06 (br s, 1H), 5.32 (s, 5H), 5.68 (m, 1H), 5.75 (m, 2H), 7.04 (m, 1H), 7.38 (ddd, J=15.12 Hz, 8.25 Hz and 1.15 Hz, 1H), 7.50 (ddd, J=14.89 Hz, 8.02 Hz and 1.15 Hz, 1H), 7.64 (d, J=8.02 Hz, 1H), 8.44 (d, J=8.25 Hz, 1H), 8.75 (d, J=2.06 Hz, 1H), 8.77 (d, J=2.06 Hz, 1H). (MS: M+H=1052.9, C₆₀H₇₇NO₁₅ requires 1052.25).

### Example 15

To test the ability of polyether ladder compounds to act as escorters, three separate comparisons were made:
1) The ability of escorter-active agent conjugates to pass across membranes of various cell types;
2) The ability of various escorter types conjugated to an active agent to pass across the membrane of a single cell type;
3) The ability of a single escorter attached to various active agents to pass across the membrane of a single cell type.

These studies include cell treatments with mixtures of the unconjugated escorter and active agents and active agents alone. The following results demonstrate the findings for the three studies described above.

### Cell Preparation, Methods and Materials

### Fluorophore-conjugated PbTx-2/Hoechst 33342 Staining Protocol

### Final Concentrations:

1:1000 Hoechst 33342 (0.1 mg/ml H-dye in H₂O stock, stored in fridge)

400 nM Fluorophore-conjugated PbTx-2, Brevenal, Brevisin* (100 μM-1 mM stocks in EtOH in fridge)
BODIPY®-PbTx-2, BODIPY®-Brevenal, BODIPY®-Brevisin
6-TAMRA-PbTx-2
Daunorubicin-PbTx-2

400 nM PbTx-2, Brevenal, and Brevisin (unconjugated, 1 mM stocks in DMSO in fridge)

400 nM BODIPY®, 6-TAMRA, Daunorubicin (unconjugated, 100 μM-1 mM stocks EtOH in fridge)

Eleven different cell lines were utilized for the cell permeability studies (CHO-K1, SJCRH30, HEK293, 184B5, MCF7, T47D, BT549, A549, DMS-114, NL20, MDCK). Four lines originated from human mammary tissue, MCF7, T47D, BT-549 (all three from the National Cancer Institute DCTD Tumor Repository) and 184B5 (ATCC CRL-8799). 184B5 cells were grown in Mammary Epithelial Growth Medium (MEGM, Lonza) supplemented with 1 ng/ml cholera toxin. The MCF7, T47D, and BT-549 cells, as well as the SJCRH30 human rhabdomyosarcoma (ATCC CRL 2061) and DMS-114 human lung small cell carcinoma (NCI DCTD TUMOR Repository) cells were grown in RPMI-1640 (ATCC) with 10% fetal bovine serum (FBS, Invitrogen) and 2 mM L-glutamine (Invitrogen). The human lung carcinoma cell line, A549 (ATCC CCL-185), and Chinese Hamster Ovary CHO-K1 (ATCC CCL-61) cells were grown in F-12K medium (ATCC) with 10% FBS. Human kidney HEK 293 (ATCC CRL-1573) and Madin-Darby Canine Kidney (MDCK, ATCC-34) cells were grown in Eagle's Minimum Essential Medium (ATCC) with 10% FBS. Normal human bronchus cells (NL20, ATCC CRL-2503) were grown in HAMS F12 (Invitrogen) with 4% FBS, 1.5 g/L sodium bicarbonate, 2.7 g/L glucose, 2 mM L-glutamine, 0.1 mM nonessential amino acids, 0.005 mg/mL insulin, 10 ng/mL epidermal growth factor, 0.001 mg/mL transferin and 500 ng/mL hydrocortisone. All media were supplemented with 100 pg/ml streptomycin and 100 units/ml penicillin (Invitrogen). All cultures were maintained in a humidified incubator with 5% CO₂ at 37° C.

For experiments, cells were seeded at a density of 10,000-20,000 cells/well in BD Biocoat poly-D-lysine-coated 96-well plates and incubated at 37° C. overnight. Cell nuclei were stained with a 0.1 pg/ml final concentration of Hoechst 33342 (Invitrogen) at least one hour prior to treatment with escorter active agent conjugates or mixtures of the individual components. 10 μl of 10× Hoechst dye in Phosphate Buffered Saline (PBS) was added directly to the growth medium in the wells, and the plate was incubated at 37° C. Cells were then treated with 10 μl of 10× solutions of escorter-active agent conjugate, an unconjugated mixture of escorter and active agent or active agent alone. Each treatment was prepared in PBS+0.4% DMSO+4% ethanol. All treatments were added directly to the growth medium to attain a final concentration of 400 nM of test article and the cells were then incubated for 1 hour at 37° C. The staining medium was then removed and cells were rinsed with 100 μl/well of Hanks Buffered Saline Solution (HBSS, Invitrogen). Cells were then photographed in 100 μl/well HBSS using a 40× (Examples 15A, 15B) or 20×(Example 15C) magnification objective. Imaging was performed on an Image Xpress Micro system equipped with an environmental control chamber warmed to 37° C. Hoechst 33342 (blue), BODIPY® (green), and 6-TAMRA/Daunorubicin (orange-red) staining were visualized using a DAPI filter, an FITC filter, and a TRITC filter, respectively. Transmitted light images were also collected to assess cell morphology.

### Example 15A

### Same Fluorophore-Polyether in Various Cell Types

In this study a BODIPY®-PbTx-2 (Example 4) conjugate was used with 11 cell types (CHO-K1, SJRH30, HEK293, 184B5, MCF7, T47D, BT549, A549, DMS-114, NL20, MDCK). Results of treatment of various cell types with the same active agent (BODIPY®) conjugated and unconjugated with polyether PbTx-2 are shown in Table 4. The level of fluorescence is shown where the highest intensity is marked “+++++” and the lowest intensity is marked “+”, “−” stands for no intensity, and ND indicates no data.

TABLE 4

| | Treatment | | | |
|---|---|---|---|---|
| | BODIPY ® − | BODIPY ® + | | |
| | PbTx-2 | PbTx-2 | | None |
| Cell Type | Conjugate | unconjugated | BODIPY ® | (control) |
| CHO-K1 | +++++ | + | − | − |
| SJCRH30 | +++++ | ++ | + | − |
| HEK293 | +++++ | − | − | − |
| 184B5 | +++++ | + | + | − |
| MCF7 | +++++ | ++ | ++ | − |
| T47D | +++++ | + | + | − |
| BT549 | +++++ | ++ | + | ND |
| A549 | +++++ | + | − | ND |
| DMS-114 | +++++ | − | − | ND |
| NL20 | +++++ | ++ | ++ | − |
| MDCK | +++++ | +++ | ++ | − |

### Example 15B

### Same Fluorophore Conjugated to Various Polyethers in the Same Cell Type

In this study, the same active agent (BODIPY®) was conjugated with three different escorters; PbTx-2 (Example 4), brevenal (Example 2) and brevisin (Example 3). Each conjugate was the tested in the same cell type (SJCRH30). Results of treatment of SJCRH30 cells with the same active agent (BODIPY®) conjugated and unconjugated with various polyethers are shown in Table 5. The level of fluorescence is shown where the highest intensity is marked “+++++” and the lowest intensity is marked “+”, and “−” stands for no intensity.

TABLE 5

| | Treatment | | | |
|---|---|---|---|---|
| | BODIPY ® − | BODIPY ® + | | |
| | polyether | polyether | | None |
| Polyether | Conjugate | unconjugated | BODIPY ® | (control) |
| PbTx-2 | +++++ | + | + | − |
| Brevenal | +++++ | ++ | + | − |
| Brevisin | +++++ | + | + | − |

### Example 15C

### Various Fluorophores Conjugated to the Same Polyether in the Same Cell Type

In this study, the same escorter (PbTx-2) was conjugated with three different active agent; Daunorubicin (Example 1), BODIPY® (Example 4) and 6-TAMRA (Example 5). Each conjugate was the tested in the same cell type (SJCRH30). Results of treatment of SJCRH30 cells with the same escorter (PbTx-2) conjugated and unconjugated with various fluorophores are shown in Table 6. The level of fluorescence is shown where the highest intensity is marked “+++++” and the lowest intensity is marked “+”, and “−” stands for no intensity.

TABLE 6

| | Treatment | | | |
|---|---|---|---|---|
| | Fluorophore − | Fluorophore + | | |
| | PbTx-2 | PbTx-2 | | None |
| Fluorophore | Conjugate | unconjugated | Fluorophore | (control) |
| Dauno- | +++++ | − | − | − |
| rubicin | | | | |
| BODIPY ® | +++++ | + | + | − |
| 6-TAMRA | +++++ | − | − | − |

Having now fully described this invention, it will be appreciated by those of ordinary skill in the art that the same can be practiced with a wide and equivalent range of compositions, modes of administration, therapeutic treatments and the like, without affecting the spirit or scope of the invention or any embodiment thereof.

1. A conjugate comprising a ladder frame polyether compound and at least one of the group consisting of biologically active compounds and research compounds, or a salt, solvate, hydrate or coordination compound thereof.

2. The conjugate of claim 1, wherein the at least one compound is a biologically active compound.

3. The conjugate of claim 1, wherein the at least one compound is a research compound.

4. The conjugate of claim 2, wherein the at least one biologically active compound is a drug or pro-drug.

5. The conjugate of claim 2, wherein the at least one biologically active compound is a pesticide.

6. (canceled)

7. The conjugate of claim 1, wherein the ladder frame polyether compound is a brevisin compound.

8. The conjugate of claim 1, further comprising one or more linkers connecting one or more of A to L.

9. (canceled)

10. (canceled)

11. A pharmaceutical formulation comprising a pharmaceutically effective amount of the conjugate of claim 1 and at least one pharmaceutically acceptable carrier.

12. (canceled)

13. A formulation for use on non-animal target species comprising an effective amount of the conjugate of claim 1 and at least one adjuvant.

14. (canceled)

15. A formulation for the control of insects comprising an effective amount of the conjugate of claim 1 and at least one adjuvant.

16. (canceled)

17. A method of improving the cellular uptake of a compound selected from the group consisting of one or more biologically active compound and research compound comprising administering the conjugate according to claim 1 to a target species.

18. (canceled)

19. (canceled)

20. A method of improving the cellular uptake of a compound selected from the group consisting of one or more biologically active compound and research compound comprising administering the conjugate according to claim 1 to a target species.

21. A method of improving the absorption cellular uptake of a compound selected from the group consisting of one or more biologically active compound and research compound comprising administering the conjugate according to claim 8 to a target species.

22. A method of treating a disease state in an animal in need of treatment comprising administering an effective amount of the conjugate according to claim 1, or a pharmaceutically acceptable salt, solvate, hydrate or coordination compound thereof.

23. (canceled)

24. (canceled)

25. (canceled)

26. (canceled)

27. (canceled)

28. A method of treating a non-animal pest selected from the group consisting of an agricultural and horticultural pest, comprising applying the formulation of claim 13.

29. (canceled)

30. A method of treating an insect infestation, comprising applying the formulation of claim 15.

31. (canceled)

32. (canceled)

33. (canceled)

34. (canceled)

35. A method for determining the effect of a biologically active molecule or a research molecule on a target species, the method comprising administering the biologically active molecule or the research molecule to the target species, as a conjugate according to claim 1.

36. A method for determining the effect of a biologically active molecule or a research molecule on tissue or cells from a target species, the method comprising contacting the biologically active molecule or the research molecule with the tissue or cells, as a conjugate according to claim 1.

37. A kit comprising a package containing a ladder frame polyether compound according to claim 1 and labeling indicating that the ladder frame polyether compound is for use in an assay for determining the effect of a biologically active molecule or a research molecule on a target species, or on tissue or cells from a target species.

38. The conjugate of claim 8, where at least one of the one or more linkers is a photolabile linker.
